(19) United States
(12) Patent Application Publication
(10) Pub. No.: US 2018/0171288 A1
(43) Pub. Date: Jun. 21, 2018

(54) METHODS OF TREATING CANCER

(71) Applicant: THE UNIVERSITY OF ROCHESTER, Rochester, NY (US)

(72) Inventors: Mark Noble, Rochester, NY (US); Brett Stevens, Commerce City, CO (US); Hsing-Yu Chen, Newton, MA (US); Jennifer Stripay, Rochester, NY (US); Christopher Folts, Rochester, NY (US); Nicole Scott, West Henrietta, NY (US)

(21) Appl. No.: 15/819,843

(22) Filed: Nov. 21, 2017

Related U.S. Application Data
(60) Provisional application No. 62/424,734, filed on Nov. 21, 2016.

(51) Int. Cl. C12N 5/00; G01N 33/574; C12N 9/10; C07K 16/30; A61P 35/00

(52) U.S. Cl. CPC C12N 5/0093; G01N 33/57492; C12N 9/104; C07K 16/30; A61P 35/00; G01N 2033/57465; C12Y 203/02; G01N 2033/57453; G01N 2033/57457; G01N 2033/57461; G01N 2033/57403

(57) ABSTRACT

Compositions and methods of treating a subject with cancer are described. The methods include administering to the subject a therapeutically effective amount of a pharmaceutical composition comprising a compound with the dual characteristics or restoring function of the c-Cbl ubiquitin ligase in cancer cells and in also worsening sublethal lysosomal dysfunction, preferably in combination with an agent capable of making cancer cells more oxidized. The pharmaceutical composition can be administered with an additional therapeutic agent or agents such as an agent that represents an accepted standard of care for the treatment of a particular type being treated. The methods can be used for treating a wide variety of different kinds of cancers, such as brain tumors, breast cancers, melanomas, breast cancers and lymphomas. The compositions and methods have the characteristics of enabling simultaneous regulation of multiple proteins known to be critical in cancer cell function, of targeting cancer stem cells and of enhancing sensitivity of cancer cells to the effects of tamoxifen and other anticancer agents.

### CROSS REFERENCE TO RELATED APPLICATION

This application claims the benefit of U.S. Provisional Application 62/424,734, filed on Nov. 21, 2016, the contents of which are hereby incorporating in its entirety.

### STATEMENT OF GOVERNMENT SUPPORT

This invention was made with government support under R01 CA214066 and F31CA177185 awarded by the National Institutes of Health. The government has certain rights in the invention.

### BACKGROUND

While advances have been made in early detection of tumors and in treatment of early-stage tumors, the ability to treat malignant cancers has not seen similar levels of improvement. There are three challenges that are particularly important to address in developing new cancer treatments. These are the challenges of tumor heterogeneity, of developing treatments that destroy or control cancer cells without causing unacceptable levels of damage to normal tissue, and of eliminating cancer stem cells.

Tumor heterogeneity occurs at two levels, both of which make successful cancer treatments more difficult to achieve. First, tumors in different patients can differ greatly in terms of the specific mutations acquired and the genetic background in which they operate. This means that even though two cancers may be classified as the same type of cancer, they differ genetically in many different ways.

At least as challenging as heterogeneity between cancers occurring in different subjects is the problem that as tumors become more malignant their internal heterogeneity (generally referred to as micro-heterogeneity) increases. By the time a tumor is malignant, cancer patients do not harbor a single disease entity. Instead, they harbor dozens or more (and perhaps many more) molecularly different cancers.

Still another challenge to developing improved cancer therapies is that killing cancer cells without killing normal cells can be extremely hard to achieve. This is a particularly difficult challenge to address, because many of the proteins and molecular mechanisms of central importance in cancer cells are also of central importance in the function of normal cells, and particularly of stem and progenitor cells. Thus, it is not surprising that most current anticancer therapies can cause unacceptable levels of damage in normal tissue.

Damage to the central nervous system (CNS) is a particular challenge associated with current cancer treatments. Systemic treatment with many different chemotherapeutic agents can cause cognitive changes, alterations in motor function and changes in CNS structure in an unacceptably high proportion of patients. Multiple studies have now shown that multiple cell types of the CNS are more vulnerable to the effects of chemotherapeutic agents than human cancer cells. Such toxicities can occur rapidly after treatment and often are durable in their effects. Moreover, in at least some cases, the damage seen weeks or months after the cessation of treatment is delayed in its onset and becomes over time even worse than that seen while treatment was ongoing. Although damage to the CNS is particularly important in respect to maintaining the quality of life of cancer survivors, damage also is seen in other tissues including, but not limited to, the gut, the hematopoietic system and the peripheral nervous system.

Still a third challenge in treating cancer is the need to eradicate a subpopulation of cells, called cancer stem cells or tumor initiating cells, that are particularly potent at forming new tumors. While there is continued discussion about the best means of prospectively identifying cancer stem cells, there is broad agreement that one of the characteristics of these cells is that they are resistant to standard anti-cancer therapies. Thus, even when a therapy is applied that results in marked tumor shrinkage, the cancer stem cells may remain. As soon as treatment ceases, a new tumor then arises, and this tumor may be even more resistant to therapies that had partial effects in their first application.

Moreover, it has become clear that cancer stem cells and normal stem cells/progenitor cells use many of the same proteins to control their division, differentiation and survival. This adds still another layer of complexity to the development of novel cancer therapies, as targeting cancer stem cells may lead to the unacceptable toxic effects on normal stem cells and/or normal progenitor cells.

Thus, there is a great need in the field of cancer research for providing improved means of identifying drugs that are more effective at controlling malignant cancers without causing unacceptable levels of damage to normal tissue; for identifying drugs that target novel molecular mechanisms for which existing standard assay approaches are not effective; to create rational combinations of drugs that enable mechanism-based control of cancer cells; and to target multiple of the proteins known to be critical in enabling cancer cell function with a single therapeutic intervention.

One theoretical, but thus far evasive, means of treating cancer that might be less damaging than existing approaches would be to harness the activities of tumor suppressor proteins (TSPs, also referred to as tumor suppressor genes). These are proteins that have the normal function of preventing aberrant cell function. TSPs are frequently mutated in cancer, with the best known example of such a TSP being the p53 protein that has been found to be mutated in almost all different types of cancers.

Developing cancer treatments based on restoring TSP function, thus harnessing the cell's own anti-cancer mechanisms for therapeutic purposes, has been of great theoretical interest for many years, particularly as inactivation of one or more TSPs is a frequent component of transformation in many different types of cancer. There has not been substantial progress towards achieving this goal in a clinically useful manner, despite the efforts of many researchers to develop such interventions.

Two challenges particularly hinder development of tumor suppressor-based therapies: (i) It is necessary not just to develop strategies for restoring normal function of TSPs—which has itself proven difficult to achieve in a clinically useful manner—but also to ensure activation of the TSP within transformed cells. (ii) These goals preferably need to be achieved with pharmacological agents to increase the likelihood that all tumor cells will be successfully targeted.

One of the TSPs that has been virtually unexplored as a therapeutic target is the E3 ubiquitin ligase c-Cbl. In normal cells, activated c-Cbl ubiquitylates and enhances degradation of specific receptor tyrosine kinases (RTKs) and other proteins important in cell division and cell survival (as summarized in FIG. 1). In contrast, in transformed cells this function of c-Cbl can be abrogated, for example by mutations that convert c-Cbl into a dominant negative oncogene or by proteins that inhibit c-Cbl activation (and in this way cause the same outcomes as mutation).

Examples of cancers in which normal c-Cbl function has been found to be inhibited include glioblastoma (GBM) cells—one of the most deadly of human tumors—due to tumor-specific complex formation with Cool-1/βpix (a PAK-interacting exchange factor also referred to as guanine nucleotide exchange factor 7). Loss of c-Cbl function leads to increased levels of multiple proteins of interest as cancer therapeutic targets, and restoration of normal c-Cbl function could thus theoretically cause decreases in levels of these same proteins. Inhibition of c-Cbl also can be found in particular types of breast cancer cells.

In the case of basal-like breast cancers, in which c-Cbl appears to be inhibited by the Cdc42 protein, challenges to identifying therapeutically useful approaches are also considerable. Although a small number of Cdc42 inhibitors have been identified, it is unclear whether they will prove useful in a clinical setting. It is also not known if inhibitors that are being pursued would have any effect on restoring c-Cbl function.

Even if it were possible to restore normal c-Cbl function in cancer cells, it is not clear that this would prove therapeutically useful for the same reasons that development of TSP therapies have been unsuccessful. For example, it may be that it is not sufficient simply to overcome the inhibition of the TSP and that it also is necessary to provide a means of activating the TSP.

Still a further challenge to the development of any cancer therapy, and particularly of TSP-based therapies, is the question of whether such an approach would be safe to implement. Because TSPs control many proteins that are important in the function of normal cells, it is not evident that even if one could activate these proteins in cancer patients in a therapeutically useful manner or whether activation can be achieved without causing unacceptable levels of damage to normal tissue. This is particularly so in the case of c-Cbl as many of the proteins that are targets of c-Cbl play important roles in the division and survival of normal cells.

Another challenge that prevents the straightforward development of therapies based on overcoming the inhibition of c-Cbl in cancer cells is a problem of broad relevance to all forms of cancer treatment, which is the need to identify patients for whom a particular therapy would be relevant. The increasing recognition of the importance of personalized approaches to cancer treatment applies to any novel treatment strategy, and the ability to identify patients for whom a therapy is appropriate is not just important in the clinical setting but also in the development of a therapy. This is because treating patients for whom a therapy is not appropriate introduces negative outcomes into a clinical study that decreases the likelihood of recognizing a statistically significant number of positive outcomes.

For therapies based on inhibiting mutant proteins, it is possible to identify candidates for treatment by gene sequencing, or other methods known to those skilled in the art, but such mutations do not appear to be important contributors to the inhibition of c-Cbl by another protein. Even in the cases in which c-Cbl function is inhibited by mutation, thus creating a dominant negative protein that is converted from TSP function to oncogene function, mutational analysis provides no benefit. This is because the need in such instances is to identify means of restoring the activation of c-Cbl, which has proven very difficult to achieve in dominant negative mutant proteins by pharmacological means.

The present invention addresses and overcomes the many obstacles that stand in the way of developing cancer treatments based on harnessing the TSP properties of c-Cbl in a clinically useful manner. The present invention provides methods for discovering agents that can be used to restore c-Cbl function in cancer cells in a therapeutically relevant manner, examples of such compounds, demonstration that such compounds provide a novel class of anticancer treatment agents in multiple respects, and methods for rational use of these compounds for the treatment of cancer and for the elimination of cancer stem cells from a mixture of cells in vitro, demonstration of the utility of such approaches in relevant treatment experiments and strategies for identifying patients for whom a particular therapy would be appropriate.

### SUMMARY

Disclosed herein are novel methods and compounds for the treatment of cancer by restoring function of the c-Cbl ubiquitin ligase, and methods of rational application of such compounds with other therapeutic agents in order to enhance cancer treatment. Agents that are capable of restoring c-Cbl function in cancer cells are referred to as c-Cbl restorative agents, or CRAs. Further disclosed are a novel class of compounds that restore c-Cbl function and enhance lysosomal dysfunction as single agents. Also provided are multiple examples of Dual c-Cbl Lysosomal Targeting Drugs, abbreviated herein DCLTDs, which represent a subset of CRAs. Together, these agents are referred to as CRAs/DCLTDs except in cases where it is necessary to distinguish agents with DCLTD activity from those that may have CRA activity but do not disrupt lysosomal function.

Disclosed also are methods of increasing the sensitivity of cancer cells to standard cancer therapies by restoring c-Cbl function and activating c-Cbl via the redox/Fyn/c-Cbl (RFC) pathway by co-exposing cancer cells to a clinically useful CRA/DCLTD and a clinically useful pro-oxidant; methods of increasing the sensitivity of cancer cells to standard cancer therapies by restoring c-Cbl function and worsening lysosomal function with CRAs/DCLTDs; methods of activating c-Cbl via the RFC pathway by co-exposing cancer cells to a clinically useful CRA/DCLTD and a clinically useful pro-oxidant; methods of enhancing the therapeutic effectiveness of standard cancer therapies by restoring c-Cbl function with a CRA and activating c-Cbl via the RFC pathway by co-exposing cancer cells to a clinically useful CRA/DCLTD and a clinically useful pro-oxidant; methods of enhancing the therapeutic effectiveness of standard cancer therapies by restoring c-Cbl function with a CRA and worsening lysosomal function with a CRA that is a DCLTD and activating c-Cbl via the RFC pathway by co-exposing cancer cells to a clinically useful CRA/DCLTD and a clinically useful pro-oxidant; methods of treating cancer by restoring c-Cbl function with a CRA and activating c-Cbl via the RFC pathway by co-exposing cancer cells to a clinically useful CRA and a clinically useful pro-oxidant; methods of treating cancer by restoring c-Cbl function with a CRA and worsening lysosomal function with a CRA that is a DCLTD and activating c-Cbl via the RFC pathway by co-exposing cancer cells to a clinically useful CRA/DCLTD and a clinically useful pro-oxidant; methods of killing cancer stem cells by restoring c-Cbl function with a CRA and activating c-Cbl via the RFC pathway by co-exposing cancer cells to a clinically useful CRA and a clinically useful pro-oxidant; methods of killing cancer stem cells by restoring c-Cbl function with a CRA and worsening lysosomal function with a CRA that is a DCLTD and activating c-Cbl via the RFC pathway by co-exposing cancer cells to a clinically useful CRA/DCLTD and a clinically useful pro-oxidant; methods of purging cancer cells from bone marrow by restoring c-Cbl function with a CRA and activating c-Cbl via the RFC pathway by co-exposing cancer cells to a clinically useful CRA and a clinically useful pro-oxidant; methods of purging cancer cells from bone marrow by restoring c-Cbl function with a CRA and worsening lysosomal function with a CRA that is a DCLTD and activating c-Cbl via the RFC pathway by co-exposing cancer cells to a clinically useful CRA/DCLTD and a clinically useful pro-oxidant.

Further provided are methods of using CRAs/DCLTDs in combination with other cancer therapies, such as chemotherapy or radiation therapy. Disclosed here are methods of assaying cancer and tumor sensitivity to compounds that are CRAs or DCLTDs. Such methods can be combined with the aforementioned methods to provide further enhanced methods of treating cancer. Also provided are methods of identifying CRAs/DCLTDs using a series of biological and/or molecular assays.

In some embodiments, the present invention provides a method of treating cancer in a patient in need thereof, comprising: (a) determining whether a patient is a candidate for CRA/DCLTD-based therapies; (b) administering one or more agents in an amount effective to restore c-Cbl function, or to restore c-Cbl function and simultaneously worsen lysosomal function and force activation of c-Cbl, and (b) providing cancer therapy to the patient. In some embodiments, the agent (b) comprises a CRA/DCLTD.

In some embodiments, the agent (b) comprises estradiol propionate, neomycin, metoprolol, methylprednisolone, dequalinium, maprotiline, meclizine, naloxone, mechlorethamine, celastrol, carboplatin, acetyltryptophan, azadirachtin, proadifen, piracetam, cyclosporine, azacitidine, simvastatin, fluphenazine, monensin, mefloquine, perphenazine, sertraline, mixtures thereof, or a pharmaceutically acceptable salt thereof.

In some embodiments, c-Cbl activation is enhanced by simultaneous treatment with agents that activate c-Cbl via the redox/Fyn/c-Cbl pathway.

In some embodiments, the cancer comprises melanoma, lymphoma, basal-like breast cancer, luminal breast cancer, glioblastoma, other gliomas, pancreatic cancer, ovarian cancer, lung cancer, head and neck cancer, bladder cancer, colon cancer, prostate cancer, bladder cancer, or esophageal cancer cells.

In some selected embodiments, the cancer therapy includes any one of surgery, radiation therapy, chemotherapy, immunotherapy, hormone therapy, stem cell transplant or combinations thereof. In some preferred embodiments, the therapeutic regimen includes radiation therapy or chemotherapy, preferably chemotherapy.

In some embodiments, the cancer therapy comprises tamoxifen, carmustine, temozolomide, irradiation, or a combination thereof.

In some embodiments, the present invention provides a method for selective ex vivo purging of cancer stem cells from a sample of bone marrow cells, comprising the steps of:

(a) harvesting bone marrow cells from a mammal;

(b) treating the harvested bone marrow cells with an agent in an amount effective to both restore c-Cbl function and worsen lysosomal dysfunction in the bone marrow cells in combination with a chemotherapeutic.

(c) implanting the treated cell back to the mammal.

In some embodiments, the present invention provides a pharmaceutical composition comprising (a) an agent in an amount effective to both restore c-Cbl function and worsen lysosomal dysfunction in cancer cells, and (b) a chemotherapeutic.

### BRIEF DESCRIPTION OF THE DRAWINGS

The accompanying drawings, which are incorporated in and constitute a part of this specification, illustrate embodiments and together with the description, serve to explain the principles of the methods and systems. Studies on glioblastoma cells and on other cancer cells are used to provide non-limiting examples of these methods and systems.

FIG. 1 illustrates some of the unique aspects of the treatments that are provided in this invention. In contrast with other approaches to the treatment of cancer, the approach provided with this invention enables targeting of the many different proteins and functions displayed in this figure (as non-limiting examples) with a single therapeutic intervention. This intervention is achieved through activation of the c-Cbl ubiquitin ligase through a non-canonical pathway referred to as the redox/Fyn/c-Cbl (RFC) pathway. Through this pathway, oxidation of cells causes sequential activation of Fyn kinase and the c-Cbl ubiquitin ligase. Oxidation of cells to activate the RFC pathway can be achieved by relying upon the increased oxidative status that is frequently found in cancer cells but can preferentially be enhanced by exposure of cells to a substance that is able to cause cells to become more oxidized. For example, in multiple examples provided, the substance used to activate the RFC pathway is tamoxifen, but it also could be any of a large variety of other agents with pro-oxidative activity, including many agents utilized in the treatment of cancer and well recognized by those skilled in the art. Activation of c-Cbl via the RFC pathway promotes degradation of multiple proteins that are direct targets of c-Cbl, and also decreases the activity, protein levels and/or mRNA levels of multiple proteins that are themselves regulated by direct targets of c-Cbl. In this manner, the treatment approach provided with this invention enables a unique targeting of multiple proteins that are required for cancer cell function with a single therapeutic intervention. Non-limiting examples of direct targets of c-Cbl that are receptors that control cell division and cell survival, and their abbreviations, include EGFR (epidermal growth factor receptor); EphA2 (ephrin type-A receptor 2); ErbB2 (erb-b2 receptor tyrosine kinase 2); FGFR-2 (fibroblast growth factor receptor-2); gp130 (glycoprotein 130); IGF-IR (insulin-like growth factor-I receptor); MET (receptor for hepatocyte growth factor); PDGFRα (platelet-derived growth factor receptor-α). Non-limiting examples of transcriptional regulators that are direct targets of c-Cbl include Notch-1, β-catenin and STAT5 (signal transducer and activator of transcription 5). A non-limiting example of an intermediate signaling proteins that is a direct target of c-Cbl includes PI3 kinase (Phosphoinositide (PI) 3-kinase). A non-limiting example of another protein that is a direct target of c-Cbl and plays an important role in cancer cell resistance to therapeutic agents is c-FLIP (FLICE-inhibitory protein). Non-limiting examples of intermediate signaling regulators and transcriptional regulators that are indirect targets of c-Cbl include NF-κB (nuclear factor kappa-light-chain-enhancer of activated B cells), SRE (serum response element) and mTOR (mammalian target of rapamycin), Nanog and STAT3 (signal transducer and activator of transcription 3), which are all downstream of various receptors that are c=Cbl targets. MGMT (O-6-methylguanine-DNA methyltransferase), a critical regulator of resistance to temozolomide and other alkylating agents, as well as resistance to irradiation, has its levels regulated by the state of activation of NF-κB, and thus is an indirect target of c-Cbl activation. In addition, the studies leading to this invention have revealed that there is a still larger number of critical regulators of cancer cell function that show decreased levels as consequence of restoring c-Cbl activation in cancer cells; non-limiting examples of such proteins include Sox 2 (SRY (sex determining region Y)-box 2), FoxM1 (Forkhead box protein M1) HSF-1 (heat shock factor-1); and HSP70 (heat shock protein 70).

FIG. 2 depicts an exemplary method for identifying agents with a desired activity by combining two or more outcomes that enable triangulation on the activity of interest. Following the principles of radio triangulation, triangulating drug screen assays work by first identifying at least two separate and measurable activities that represent divergent outcomes of activating or inhibiting the enzyme or transcription factor or receptor(s) of interest. These outcomes must represent independent outcomes. As such, these activities cannot represent a linear signaling outcome such that activity 2 depends upon activity 1 as such a linear outcome would not provide two independent vectors. Instead, triangulating drug assays are carried out by employing activities that are dependent upon the protein/activity of interest but are not dependent upon each other. In a preferred embodiment of this triangulating drug discovery assay, a third activity outcome is included that eliminates spurious outcomes that may influence assay outcome in ways that do not reflect the activity for which the screen is being conducted. Still additional activities may be added to further narrow the range of possible means of achieving the desired outcomes. FIG. 2A provides a simplified summary of signaling from a receptor tyrosine kinase. Ligand is shown bound to a receptor (green) which becomes activated (indicated by triangles with the letter “P”, which stands for phosphorylation. The activated receptor further activates downstream enzymes ERK1/2 and Akt. These in turn activate the transcription factor SRE (serum response element) and NF-κB. FIG. 2B demonstrates activation of the redox/Fyn/c-Cbl pathway. Oxidation, for example by exposure of cells to tamoxifen, causes activation of Fyn kinase. This in turn phosphorylates and activates the c-Cbl ubiquitin ligase. Activated c-Cbl attaches ubiquitin (ovals with the letters “Ub”). Ubiquitinylation of a receptor targets it for degradation (FIG. 2C), which causes decreases in activity of downstream enzymes (Erk1/2 and Akt) and transcription factors (SRE and NF-κB). FIG. 2D summarizes how this information is used to create a triangulating drug screen. Activation of c-Cbl leads to decreased activity of SRE and NF-κB, as detected in standard reporter-promoter assays. In addition, analysis of transcriptional activity of AP-1 is used to screen out compounds that are transcriptional inhibitors by selecting for compounds that do not cause decreases in AP-1 activity.

FIG. 3 provides a non-limiting example of the triangulating drug screen. In this example of the present invention, the triangulating drug screen was utilized to identify compounds that have the unexpected activity of restoring the function of c-Cbl in cancer cells using as a starting library the NIND-II library composed predominantly of drugs either approved for clinical use or in clinical studies. This library contains 1040 compounds, and results illustrate the ability of the triangulating screen to identify compounds with the desired activity.

FIG. 4A depicts the increase in tamoxifen sensitivity in GBM cells exposed to mapratoline (MPT). FIG. 4B depicts blockage of sensitivity increase by co-exposure of cells to N-acetyl-L-cysteine (NAC), a potent anti-oxidant. FIG. 4C depicts the killing of cells by the combination of MPT+TMX is dependent on activation of c-Cbl, and is blocked by shRNAi-mediated knockdown of c-Cbl.

FIG. 5 depicts the increase in tamoxifen sensitivity in BLBC cells exposed to sertraline (SRT).

FIG. 6A depicts the increase in tamoxifen sensitivity in tamoxifen-resistant luminal breast cancer cells exposed to mapratoline (MPT). FIG. 6B depicts increases in c-Cbl phosphorylation that are caused by co-exposure of cells to MPT+TMX, but are not caused by exposure to either agent on its own. FIG. 6C depicts that tamoxifen resistant (TMXR) luminal breast cancer cells do not show increases in c-Cbl phosphorylation in response to exposure to the chemical pro-oxidant tert-butyl hyrdroxide (TertB). In parental control MCF7 cells, which are sensitive to tamoxifen, exposure to TertB causes c-Cbl phosphorylation and this is prevented by co-treatment of cells with the ant-oxidant N-acetyl-L-cysteine (NAC). Thus, acquisition of tamoxifen resistance is associated with inhibition of oxidation-induced activation of c-Cbl.

FIG. 7 depicts selection for tamoxifen resistance is associated with the inhibition of oxidation-mediated activation of c-Cbl, the appearance of a complex between c-Cbl and Cool-1/βpix and increases in Cool-1/βpix phosphorylation. FIG. 7A depicts the effect of tamoxifen against standard MCF-7 and TMX-resistant (TMXR) cells (obtained by treating MCF-7 cells with 1 μM tamoxifen for six months. Unlike their parental cells, TMXR cells continue to divide in the presence of 1 μM TMX (a dose that is cytostatic but not lethal for the MCF-7 and T47D cells used in these experiments; FIG. 7A). TMXR cells expressed ERα; thus, TMX resistance was not due simply to ERα loss (not shown). FIG. 7B depicts that exposure to the pro-oxidant tert-butylhyroxide increased c-Cbl phosphorylation in parental MCF-7 or T47D cells, but not in TMXR derivatives of these cells. FIG. 7C depicts levels of c-Cbl targets (EGFR and HER2) were increased in TMXR cells. FIG. 7D depicts c-Cbl/Cool-/pix complexes in TMXR cells. Parental TMX-sensitive cells did not contain these complexes.

FIG. 8 demonstrates that MPT restores TMX-induced c-Cbl phosphorylation in GBM cells. This example demonstrates the ability of CRAs/DCLTDs to restore the ability of TMX to enhance phosphorylation of c-Cbl via the redox/Fyn/c-Cbl pathway, and the importance of exposing cells to both a CRA/DCLTD and a pro-oxidative stimulus is provided. Co-treatment of glioblastoma cells with MPT enabled TMX-induced c-Cbl phosphorylation (FIG. 8A). As predicted if activation of c-Cbl was RFC pathway-mediated, such activation was inhibited by N-acetyl-L-cysteine (FIG. 8B) (NAC, a glutathione pro-drug and anti-oxidant), as also seen in our studies on BLBC cells.

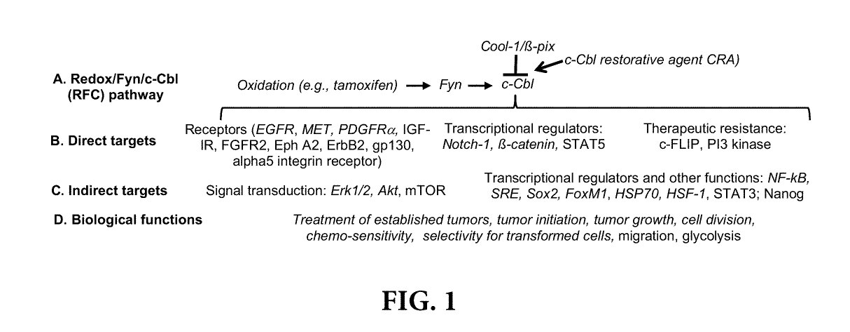

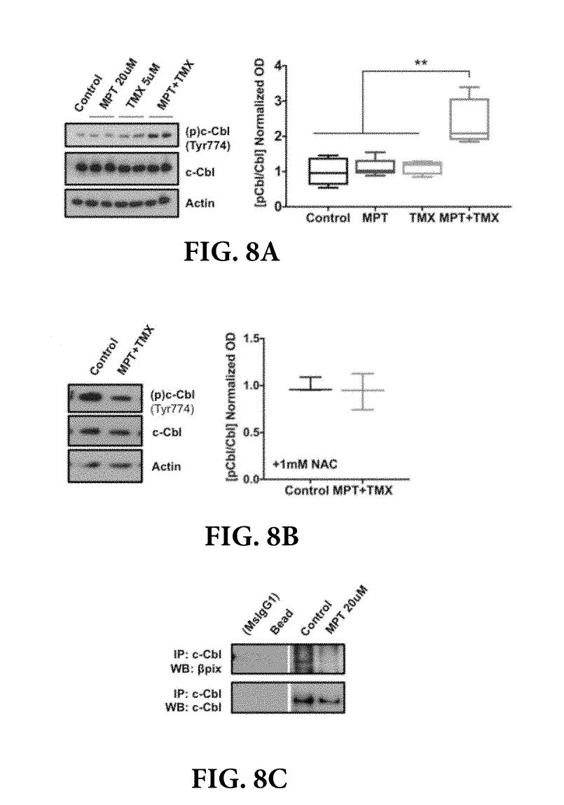

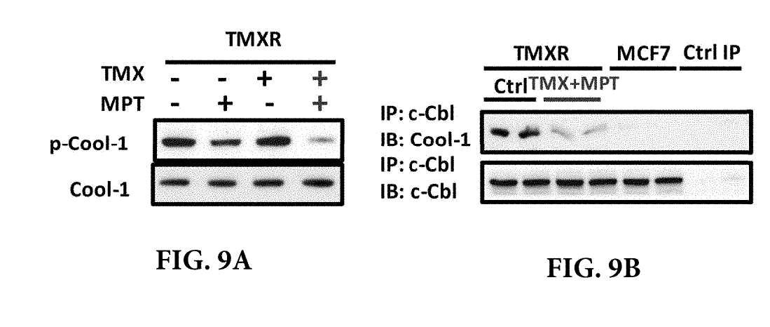

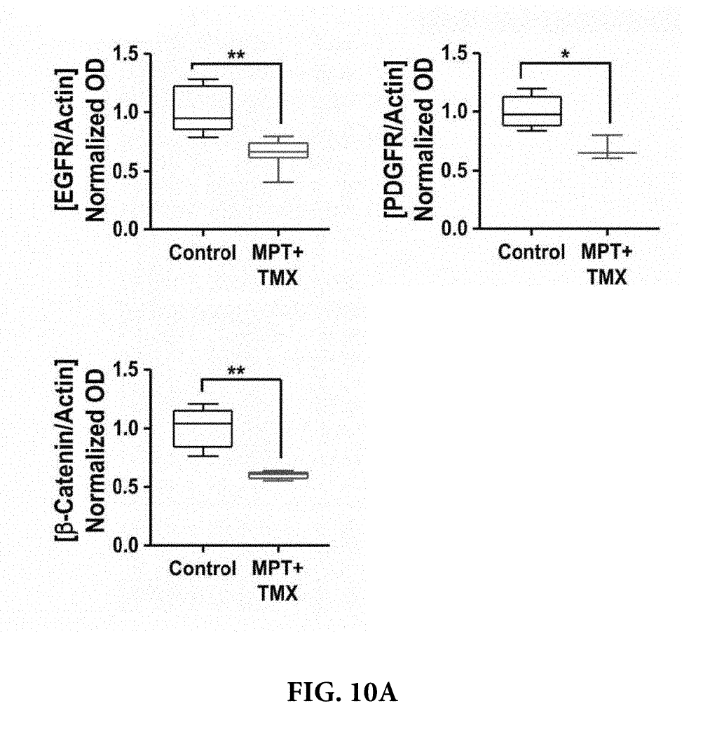

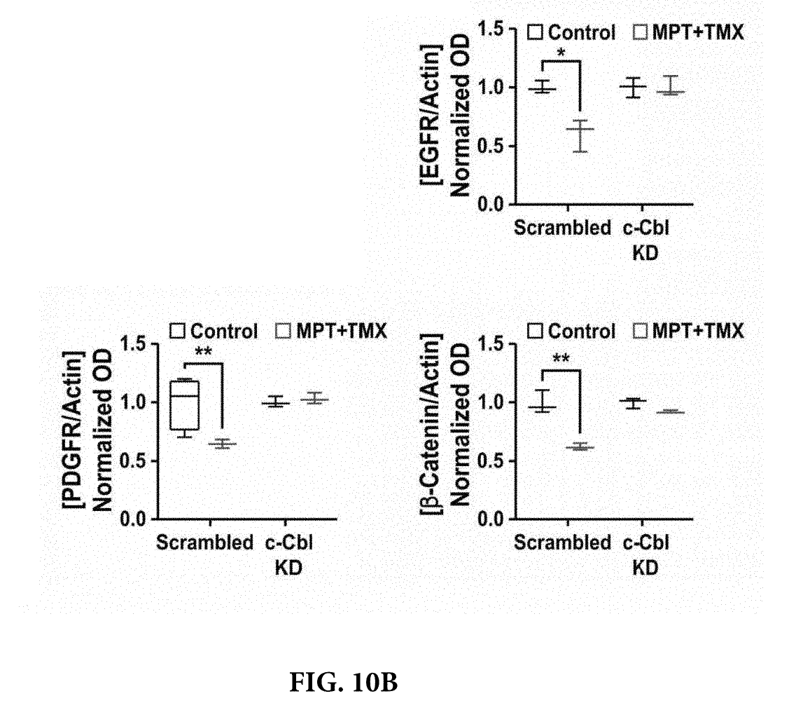

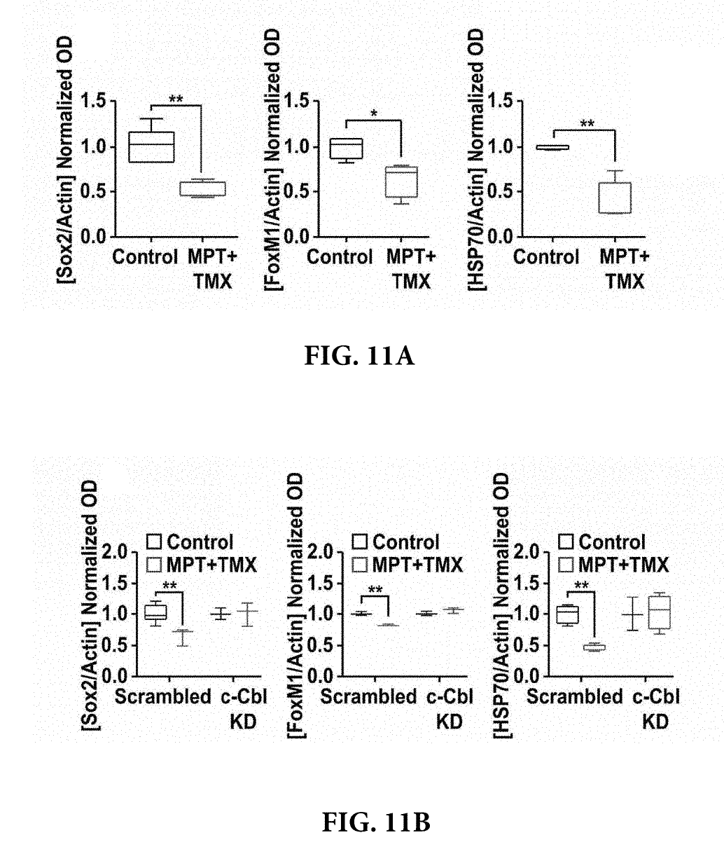

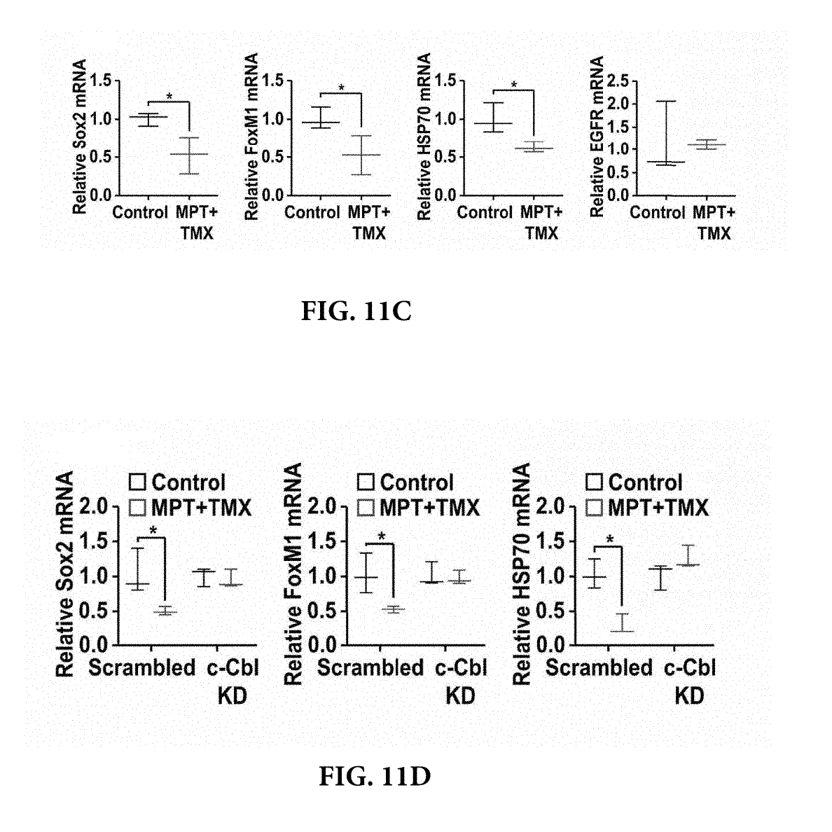

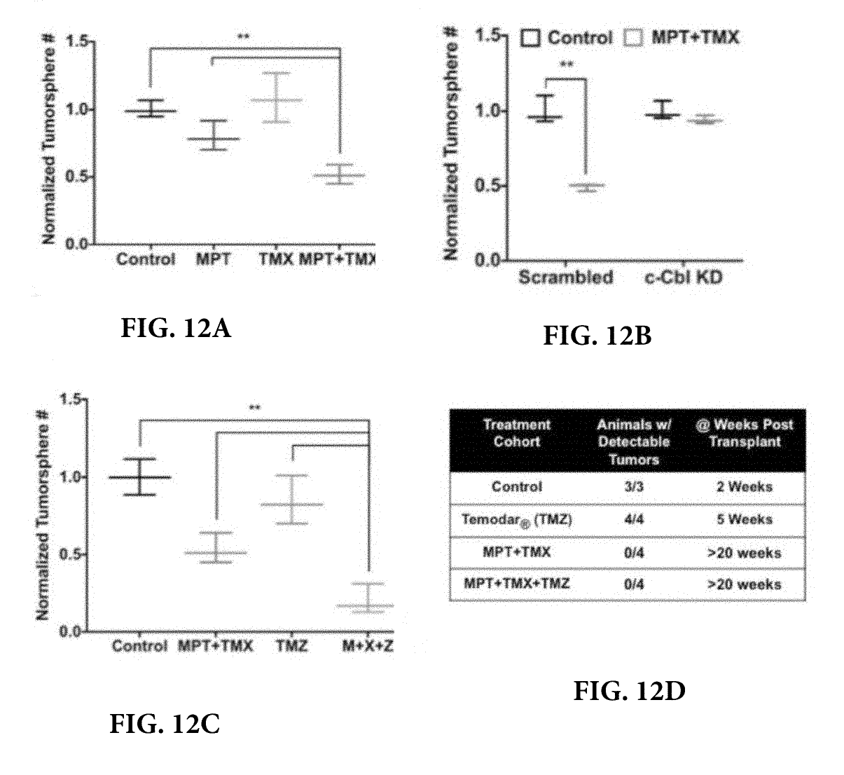

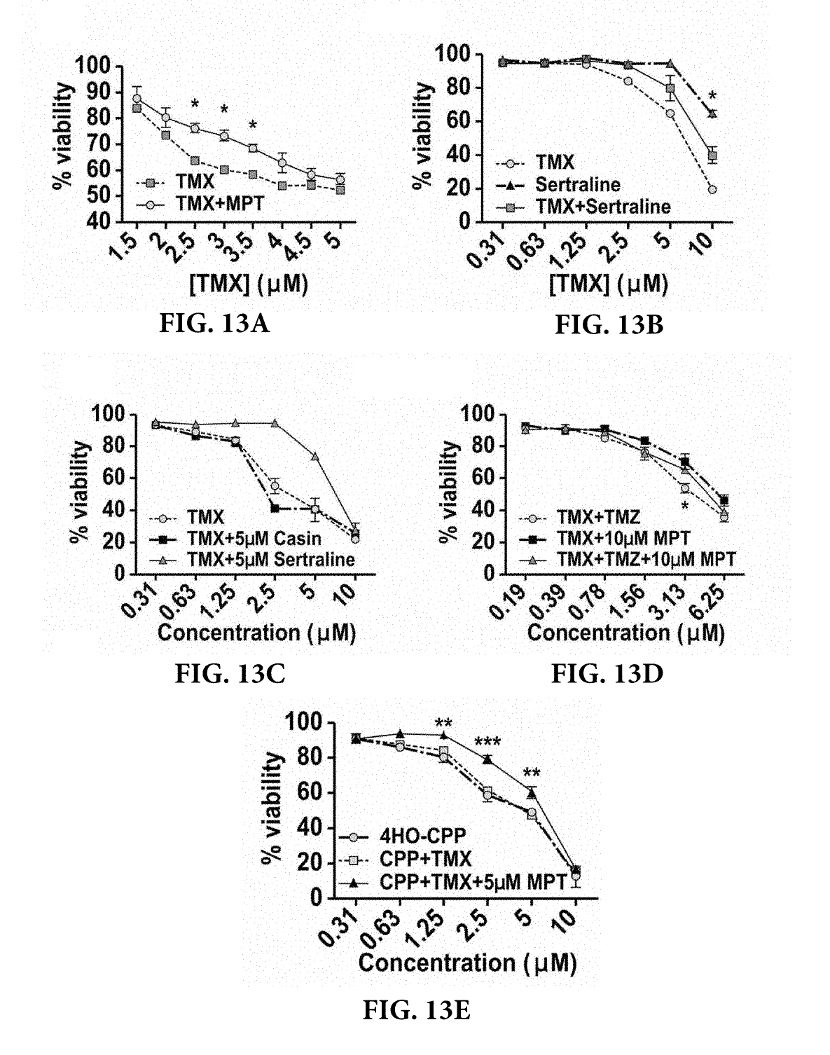

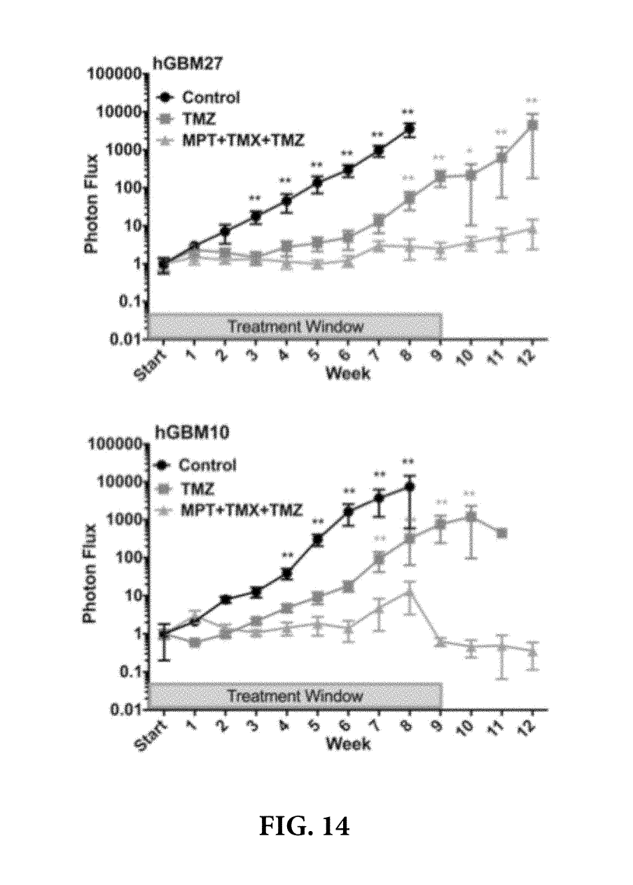

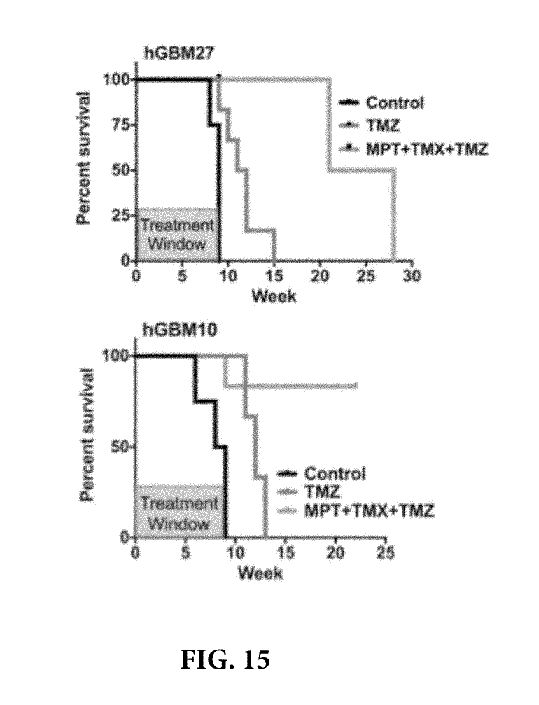

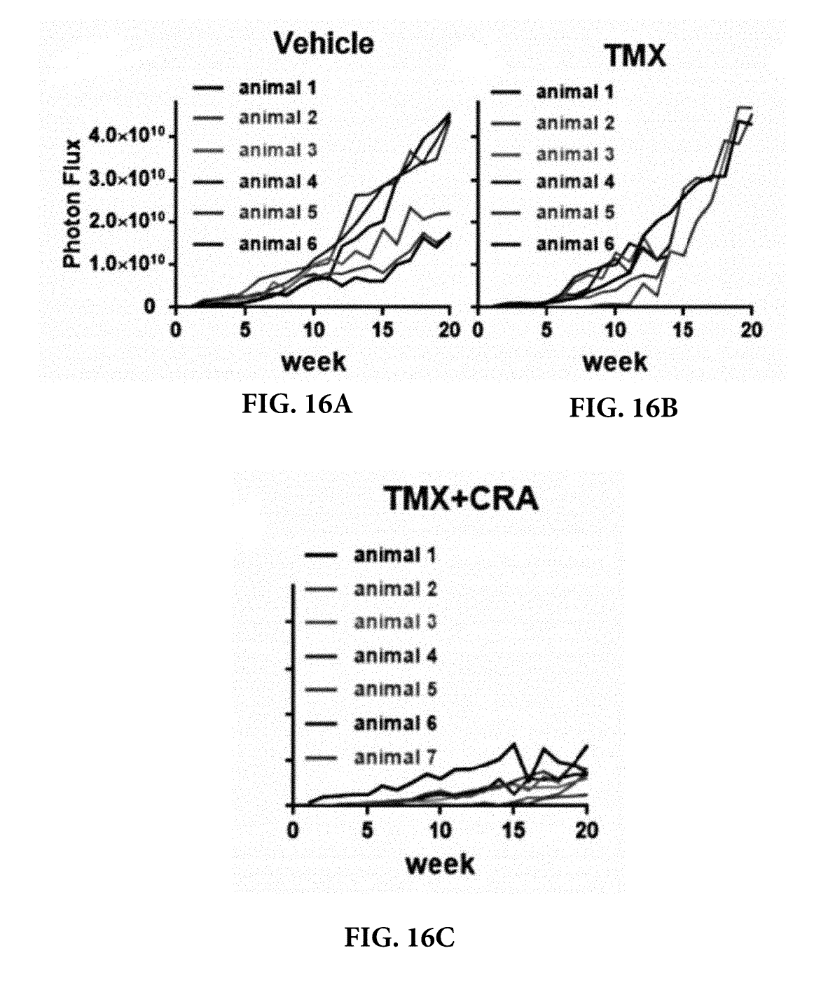

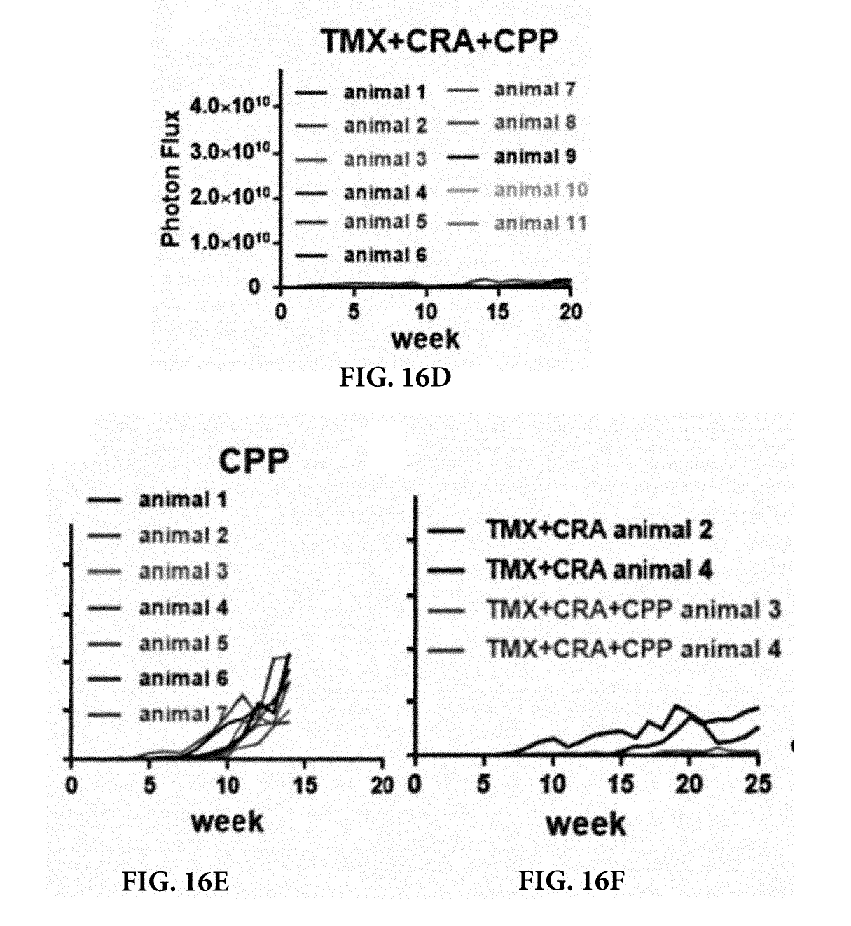

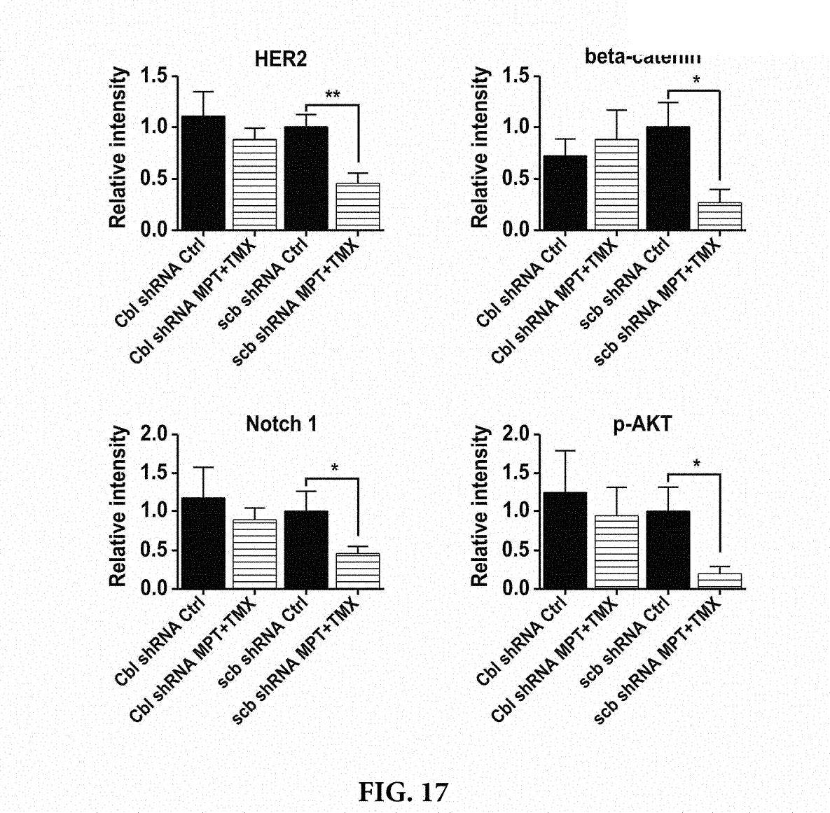

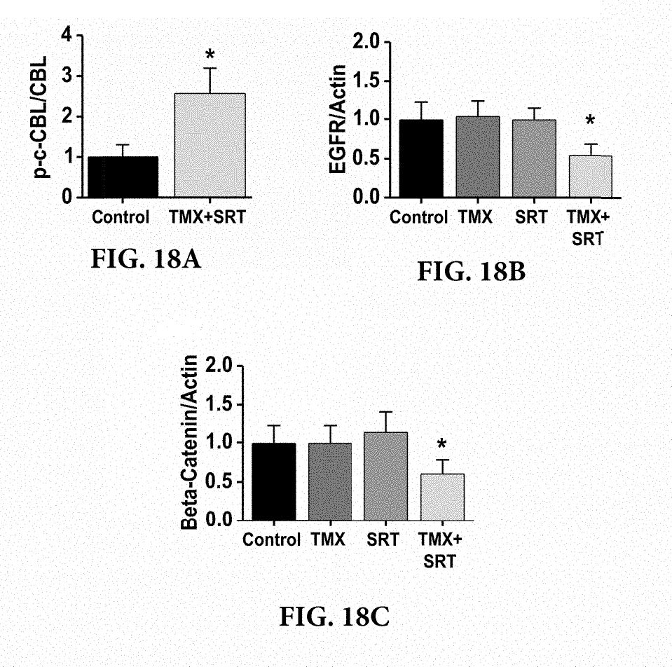

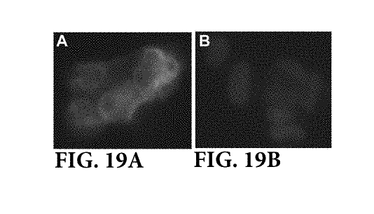

We next examined the effects of MPT on the only demonstrated mechanism by which c-Cbl activation is inhibited in the GBM cells used in our studies, which is due to tumor-specific complex formation with Cool-1/βpix. MPT exposure had the novel effect of causing marked reductions in levels of the complex between c-Cbl and Cool-1/βpix. Lysates of treated and untreated cells were immuno-precipitated with anti-c-Cbl antibody followed by immunoblot analysis for Cool-1/βpix expression. MPT-treated GBM cells showed a notable reduction in levels of complex-bound Cool-1/βpix as compared to untreated controls [FIG. 8C].

FIG. 9 depicts co-treatment of glioblastoma cells with MPT+TMX decreasing the ability of Cool-1/βpix to inhibit c-Cbl. FIG. 9A demonstrates that treatment with maprotiline (MPT) on its own causes a decrease in levels of Cool-1/βpix phosphorylation as detected with the Millipore anti-phospho-Cool1/βpix antibody that identifies phosphorylation on Serine340. Decreases in Cool-1/βpix phosphorylation are increased by exposure of cells to the combination of MPT+TMX. FIG. 9B demonstrates that exposure to MPT+TMX decreases the level of the inhibitory complex between c-Cbl and Cool-1/βpix. In the upper gel, c-Cbl is immunoprecipitated and the gel is probed with an antibody to total Cool-1/βpix, and treated cells have markedly decreased amounts of Cool-1/βpix complexed with c-Cbl. The lower gel demonstrates that this is not due to changes in amounts of c-Cbl in the cells. This gel also shows that parental MCF7 cells, which are tamoxifen sensitive, contain no complexes between c-Cbl and Cool-1/βpix.

FIG. 10A depicts that treatment of GBM cells with MPT+TMX caused decreases in levels of the c-Cbl targets of EGFR and β-catenin, as well as platelet-derived growth factor receptor-α (PDGFRα). As predicted by the redox/Fyn/c-Cbl hypothesis of c-Cbl activation, decreases were less marked when cells were exposed to MPT on its own. Moreover, these decreases were blocked by c-Cbl knockdown (FIG. 10B), demonstrating the dependence of the observed effects on the ability to activate c-Cbl.

FIG. 11 depicts that treatment of GBM cells with MPT+TMX causes decreases in levels of proteins, and their mRNAs, that are not direct c-Cbl targets. Treatment with MPT+TMX also caused decreases in levels and/or activation of other proteins that are thought to be critical in cancer cell function but which are not direct targets of c-Cbl. There are two groups of such proteins, ones in which the connection to a c-Cbl target can be reasonably surmised based on prior research and ones for which the linkage to c-Cbl is currently unknown.

FIG. 11A shows that treatment with MPT+TMX also caused decreases in levels of FoxM1, Sox2, heat-shock factor 1 (HSF-1), and heat shock protein 70 (HSP70), which are not thought to be c-Cbl targets and for which the linkage to targets of c-Cbl is currently unknown. These decreases were c-Cbl dependent, as shown in FIG. 11B. Such an outcome is both unprecedented and of great scientific interest, as research efforts have been established to find inhibitors of each of these single proteins, but there is no prior demonstration of any intervention that enables targeting of the group of them with a single therapeutic intervention. Moreover, these changes in indirect c-Cbl targets are associated with c-Cbl-dependent decreases in their levels of mRNA, something that was not seen for the direct c-Cbl target of the EGFR (FIG. 11C, FIG. 11D).

FIG. 12 demonstrates that treatment with MPT+TMX eliminates GBM TICs, and the efficacy of this treatment is increased still further by addition of temozolomide. It is increasingly recognized that cancers contain a subset of cells that are particularly important in tumor generation. These cells are called tumor-initiating cells (TICs, which also are referred to as cancer stem cells). Elimination of TICs is one of the central goals of ongoing research on new cancer therapies, but the precise identity of TICs in different tumors remains controversial. Nonetheless, it is generally agreed that TICs are more resistant to cancer treatments, such as chemotherapy and irradiation, than are the other cells that comprise the tumor.

FIG. 12A demonstrates that treatment with DCLTD-based therapies eliminates TICs. For example, GBM cells from four different cell lines were exposed to sublethal combinatorial concentrations of MPT+TMX for 5 days in vitro, at concentrations that kill about ˜10% of total cells. This treatment was associated with decreases in formation of adhesion independent spheres of cells, called tumorspheres, which are a surrogate marker of TIC function. Decreases in tumorsphere formation were c-Cbl dependent, as shown in FIG. 12B. If temozolomide was added to the mixture of MPT+TMX, elimination of tumorsphere-forming cells was even more effective, as shown in FIG. 12C. After treatment, 500,000 live cells were transplanted intra-cranially in NSG mice. Cells treated with saline or with temozolomide (TMZ, the current first-line treatment for glioblastoma) generated tumors within three weeks, as detected by photon emission from luciferase-expressing cancer cells, while cells treated with MPT+TMX showed no tumor generation even after 5 months (FIG. 12D). In contrast with the effects of MPT+TMX in TICs, treatment with temozolomide (TMZ, the frontline treatment for glioblastoma), had no apparent effects on TICs (FIG. 12D). This was not due to a protective effect of TMZ, as the combination of [MPT+TMX+TMZ] was as effective as [MPT+TMX] at eliminating TICs.

FIG. 13 demonstrates that treatment with CRAs/DCLTDs does not increase sensitivity of OPCs to standard anti-cancer agents. One of the most challenging aspects of developing effective cancer treatments is that most anticancer treatments have the problem of being able to cause great levels of toxicity to the normal cells of the body. Thus, it is a desirable (but rarely achieved) feature of any new cancer treatment to be able to increase toxicity for cancer cells without causing at the same time increases in toxicity for normal cells. This is a particularly difficult problem to solve in the context of cells of the central nervous system, as many of these cells are inherently more sensitive to anticancer agents than are cancer cells themselves. Previous studies have shown that one of the cell types of the brain that is most sensitive to the adverse effects of multiple chemotherapeutic agents are the oligodendrocyte progenitor cells (OPCs) that give rise to the myelin-forming oligodendrocytes of the central nervous system. Due to the high degree of sensitivity of OPCs to anticancer treatments, they provide a preferred cell type for use in evaluating whether new treatments exhibit desirable levels of specificity for cancer cells.

Analyses conducted on OPCs isolated from the developing human central nervous system demonstrated that MPT did not increase the sensitivity of these progenitor cells to tamoxifen (FIG. 13A). This is entirely unexpected, as the redox/Fyn/c-Cbl pathway was discovered in oligodendrocyte progenitor cells, and the most likely outcome of restoring c-Cbl function in GBM cells would be thought to the make oligodendrocyte progenitor cells and GBM cells equally sensitive to tamoxifen.

This outcome is not solely the property of MPT. FIG. 13B shows that treatment with sertraline (a CRA/DCLTD identified in studies on basal-like cancer cells rather than glioblastoma cells) also does not increase sensitivity of OPCs to TMX.

The ability to selectively increase the toxicity of TMX in cancer cells and not in vulnerable primary cells is a general property of CRAs/BLBCs. As shown in FIG. 13C, the experimental Cdc42 inhibitor CASIN also does not increase the vulnerability of OPCs to TMX. This is so even though CASIN does not provide the apparent protective activity that appears to be provided by sertraline, indicating that the selective effects of CRA/DCLTD treatment represent an important new general principle.

Even more important, the selective effects of treatment with CRAs/DCLTDs are not limited to TMX. FIG. 13D demonstrates that treatment with MPT, and even treatment with MPT+TMX does not increase the sensitivity of OPCs to temozolomide (TMZ, the front-line treatment for glioblastoma). It also does not increase the sensitivity of OPCs to 4-hydroxycyclophosphamide, a chemotherapeutic agent used in the treatment of multiple different cancers, including breast cancers, as shown in FIG. 13E.

FIG. 14 demonstrates that treatment with MPT+TMX+TMZ decreases rate of tumor growth as compared with TMZ. ERα-independent effects of TMX have been of sufficient interest to have led to clinical studies on the use of high dose TMX in treatment of GBMs, other gliomas, and a dozen other types of cancers. In all of these studies, however, there is no rational exploitation of the activities of TMX as a pro-oxidant. Moreover, none of the outcomes in these prior studies have been sufficiently compelling as to make the use of TMX of anything other than experimental interest thus far. Our results suggest this failure of TMX to provide benefit may be due to inhibition of c-Cbl, as our studies demonstrate that such inhibition prevents the estrogen receptor-α-independent activities of TMX that kill cancer cells.

As an example of the unexpected potency of CRA/DLCTD-based therapies, treatment with a DCLTD+TMX+TMZ was used to treat established GBMs growing intracranially in immune-deficient mice. Mice were treated by oral gavage with saline, TMZ, or MPT+TMX+TMZ, as all drugs in this group are orally available. Mice were treated, in a regimen of 5 days on/2 days off, until all saline-treated mice were dead (at 9 weeks after treatment initiation). All drugs were applied at clinically relevant exposure levels.

FIG. 14 depicts the administration of MPT+TMX to GBM in standard intracranial xenograft models that mimic clinically relevant elements of GBM disease course and treatment. As TMZ is the leading standard of care therapy for GBM patients, our treatment regimen also included this agent. Following stereotactic intracranial transplantation of 500,000 luciferase-expressing GBM cells, tumors were allowed to grow until observable by non-invasive bioluminescence imaging, which occurred within 2 weeks following transplant. Tumor-bearing mice were then randomly assigned to treatment cohorts of vehicle (saline) control, clinically relevant doses of TMZ (5 mg/kg), or the combination of MPT (35 mg/kg), TMX (4.2 mg/kg) and TMZ (5 mg/kg). TMZ was applied at the lowest dosages used in mice, which is about one-fifth of the standard maintenance dose received by patients (based on body surface area conversion formulas), and TMX concentrations were consistent with daily dosages in breast cancer patient populations, and only 20-25% of the concentration commonly used in high-dosage applications. The concentration of MPT used was three times higher than a patient-equivalent antidepressant dose, but well within the safely tolerated range (Mayo Clinic, www.mayoclinic.org/drug-supplements). As all drugs are orally available, they were delivered by oral gavage using a treatment regimen of 5 days on, 2 days off in an effort to mimic potential patient dosing schedules. Drugs were administered for 9 weeks (the average moribund time point for vehicle-treated mice).

For both hGBM27 or hGBM10 transplants, tumor growth was rapid in vehicle-treated mice and treatment with TMZ only transiently suppressed such growth. Increases in tumor size even during TMZ treatment were marked. For hGBM27 cells, tumor size (based on photon flux) began to increase beginning in the 4^{th} week of TMZ treatment. At the end of 9 weeks of treatment, tumor size had increased >100-fold (p<0.01) and at 12 weeks (i.e., 3 weeks after cessation of treatment) tumor size in TMZ-treated mice had increased >5000-fold (p<0.001. Results obtained with hGBM10 cells were similar, with a 500-fold increase in tumor size observed at 12 weeks following the initiation of TMZ treatment. In addition, the rate of tumor growth observed after tumor recurrence in TMZ-treated mice was similar to that seen in saline-treated mice, much as occurs in patients treated with TMZ.

TMZ treatment transiently suppressed tumor growth, but re-initiation of rapid tumor growth was apparent while treatment was ongoing (as often occurs in patients); in contrast, treatment with MPT+TMX+TMZ showed complete suppression of tumor growth throughout the time of treatment (FIG. 14), and this lack of tumor growth was maintained for several weeks or more after treatment was terminated.

FIG. 15 demonstrates that treatment of GBMs in vivo with MPT+TMX+TMZ increases survival more effectively than treatment with TMZ alone. As an example of the therapeutic efficacy of DLCTD-based therapies, immunodeficient NSG (NOD− Scid− gamma) mice were transplanted intrancranially with 500,000 luciferase-expressing human GBM cells, derived from three different GBM patients. In all experiments, tumors were allowed to grow until they were clearly visible by non-invasive imaging with an Advanced Molecular Imager (AMI; Spectral Instruments), which generally took two-three weeks. Mice were then assigned to different treatment groups, with a distribution that ensured equal representation of different tumor sizes in each group.

Mice were treated via oral gavage to administer treatment, as MPT, TMX and TMZ are all orally available. Mice treated with saline showed continuous tumor growth and died at nine weeks, at which time all treatments were stopped. TMZ treatment caused a transient suppression of tumor growth but tumor growth was evident by the seventh week of treatment. This is an outcome that is essentially identical to treatment of patients, for whom tumor recurrence is frequently seen during treatment of TMZ.

TMZ-treated mice had a median survival time of 11.5 weeks and 12 weeks in the hGBM27 and hGBM10 cohorts, respectively, an average 30% increase in lifespan over the median 9 week survival of saline-treated mice. This increase in lifespan was similar in extent to that seen in patients, in which standard TMZ treatment appears to increase lifespan by 20-30% beyond that achieved with other treatment approaches. In contrast, in mice treated with [MPT+TMX+TMZ], we observed an >180% increase in lifespan beyond that of saline-treated mice, a 5-fold greater improvement over that achieved with TMZ by itself despite TMZ being the first-line drug for GBM treatment. Median survival reached 24.5 weeks in the GBM27 cohort and all-but-one of the GBM10 cohort animals survived without any detectable tumor burden out to 22 weeks, at which point they were sacrificed and censored from the study (hGBM27 p=0.0031, hGBM10: p=0.0397).

FIG. 16 demonstrates that treatment of TMX resistant luminal breast cancers in vivo with a DCLTD (MPT) and a pro-oxidant (TMX) suppresses tumor growth, and suppression of tumor growth is even more effective when ultra-low dose cyclophosphamide is included in the treatment regimen. Another example of the general utility of treatment with a CRA/DCLTD with a pro-oxidant to offer clinically relevant benefits is offered by studies of luminal breast cancer cells selected in vitro to be resistant to treatment with TMX.

Studies on TMX-resistant luminal breast cancer cells focused on cyclophosphamide (CPP) as a standard chemotherapeutic agent. CPP is an alkylating agent used in treating TMXR breast cancer. We started in vivo studies with a CPP dose of 3 mg/kg (which is considerably below common murine doses of 10-100 mg/kg and represents a human dose equivalent of ˜0.25 mg/kg (based on FDA body surface area conversion guidelines), about 25% of standard human low dose oral usage). This starting dose was chosen based on our concerns with developing treatments less toxic than existing approaches, and on predictions of the effects of MPT+TMX treatment-induced decreases in levels and/or activity of proteins important in chemoresistance. MPT and TMX were applied at clinically relevant dosages. For MPT (35 mg/kg), the daily clinical dose in mice is 15-30 mg/kg (maximum=45 mg/kg). For TMX (4.2 mg/kg), the mouse equivalent of the daily dose for standard LBC treatment is 4-7 mg/kg, and would be increased for high dose applications. To compare outcomes with our findings on GBM cells, mice in this first group were treated with saline, TMX, [MPT+TMX], or [MPT+TMX+CPP] (oral gavage, 5 days on/2 days off).

In the experiments depicted in FIG. 16, immune-deficient NSG mice were transplanted with 500,000 luciferase-expressing human TMXR MCF7 cells in the mammary fat pads. After tumors were visible by non-invasive imaging (generally 1 to 2 weeks), mice were assigned to treatment groups, with equal representation of tumor sizes in each group. FIG. 16 demonstrates the benefit of treatment with the novel approach of the present invention. Tumors grew rapidly in saline-treated mice (FIG. 16A), and also in TMX-treated mice (FIG. 16B), thus confirming the tamoxifen resistance of these tumors. Treatment with MPT+TMX suppressed tumor growth (FIG. 16C), demonstrating that the effects of treatment with the combination of a CRA/DCLTD and TMX differs from markedly from the effects of treatment with TMX alone.

Treatment with the combination of MPT+TMX+CPP completely suppressed tumor growth in vivo (FIG. 16D), despite the fact that CPP levels were only a fraction of the lowest standard doses used in the clinic. Suppression of growth was not simply due to the effects of CPP, as treatment with CPP alone already have initiated tumor growth midway through treatment (FIG. 16E).

Treatment effects of MPT+TMX+CPP seem to be durable, as predicted from the observations that this treatment effectively eliminates tumor initiating cells. FIG. 16F shows outcomes at 5 weeks after the cessation of treatment, with no tumor recurrence. These observations raise the possibility that this treatment is able to control the growth of a type of breast cancer, resistant to hormone therapies, for which current therapies are inadequate.

FIG. 17 depicts that treatment with MPT+TMX causes decreases in multiple c-Cbl targets and other proteins critical in cancer cell and TIC function in TMX resistant luminal breast cancer cells. As already discussed in relation to GBM cells, restoration of c-Cbl function can decrease levels and/or activity of multiple proteins important in cancer cell growth. This outcome is a general characteristic of restoring c-Cbl function in cancer cells in which c-Cbl is inhibited, as indicated also by studies on TMX-resistant LuBC cells treated with MPT+TMX (FIG. 17). MPT+TMX decreases levels of HER2, EGFR, and β-catenin as examples of direct c-Cbl targets. Examples of indirect targets of c-Cbl that also are decreased by treatment of TMX resistant LuBC cells with MPT+TMX include decreased levels of phosphorylated Erk1/2 (not shown) and Akt (FIG. 17), two downstream targets of activated RTKs that decrease in activity when c-Cbl is activated via the RFC pathway. Moreover, as seen following treatment of glioblastoma cells with MPT+TMX, we also found decreased levels of Sox2 (not shown), a transcription factor critical in TIC function. All outcomes required c-Cbl function, and were prevented by c-Cbl knockdown. Moreover, all outcomes required treatment with MPT+TMX, and were not caused by treatment with but either drug alone (not shown).

FIG. 18 demonstrates that treatment of basal-like breast cancer cells with sertraline+TMX causes decreases in multiple c-Cbl targets critical in cancer cell and TIC function. The ability of CRA/DCLTD-based therapies, combined with RFC-pathway mediated activation of c-Cbl to cause decreases in levels of multiple c-Cbl targets in multiple cancers, and with different types of CRAs/DCLTDs, is supported by studies on the effects of treatment with sertraline (SRT)+TMX on c-Cbl and two c-Cbl targets in BLBC cells (FIG. 18A). Levels of phosphorylated c-Cbl were increased by treatment with HER2, Notch-1 and beta-catenin levels and activation of the indirect target (p-AKT) fare decreased by treatment with 5 uM MPT+5 uM TMX for 6 hours in scrambled knockdown cells (FIG. 18B, FIG. 18C). This effect was abolished in c-Cbl knockdown cells.

FIG. 19 demonstrates that it is possible to identify cells with a c-Cbl complex by immunostaining with antibodies against a phosphorylated amino acid (Serine 340) on Cool-1/βpix. Here evidence is provided that staining of cells with an antibody against a phosphorylated serine residue on Cool-1/βpix can be used to identify glioblastoma cells in which c-Cbl is inhibited by complex formation with Cool-1/βpix FIG. 19A). Treatment with MPT (which decreases the levels of the inhibitory complex between c-Cbl and Cool-1/βpix) decreases the level of staining with this antibody directed against Serine340 of Cool-1/βpix FIG. 19B).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes¬ from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Throughout the description and claims of this invention, the term “restoring c-Cbl function” means restoring the ability to activate c-Cbl and, through this activation, attach ubiquitin to proteins that are targets of c-Cbl and thus promote their degradation. “Activating c-Cbl” refers to providing a cell with an oxidative stimulus to cause (or enhance) c-Cbl to perform its normal regulatory function of causing degradation of proteins normally targeted by this enzyme As used herein, a c-Cbl inhibited cell is one that will exhibit increased c-Cbl function upon treatment with a CRA (or DCLTD). As used herein, a c-Cbl responsive cell is one that will exhibit increased c-Cbl function upon treatment with an agent that increases cellular oxidative status.

Throughout the description and claims of this specification, “c-Cbl restorative agent” or “CRA” means a pharmacological compound, mRNA, shRNAi or such other agents as will be apparent to those skilled in the art that when applied to a cancer cell in which activation of c-Cbl is inhibited, the CRA restores the ability of the cell to activate c-Cbl. In this specification, normal function of c-Cbl may be inhibited due to cellular expression and/or activation of a protein that is able to directly or indirectly inhibit c-Cbl or due to cellular expression and/or activation of a protein, an mRNA or a microRNA that inhibits the expression of c-Cbl. A compound that is defined as a CRA may exhibit only the ability to restore c-Cbl function or may also have other activities. A preferred CRA is one that is suitable for therapeutic use in the treatment of cancers in which normal c-Cbl function is inhibited.

Throughout the description and claims of this specification, “Dual c-Cbl Lysosomal Targeting Drug” or “DCLTD” means a pharmacological compound that, in a single compound, has the ability to restore c-Cbl function and also has the ability to worsen lysosomal dysfunction. Compromising of lysosomal function may occur by multiple means. Non-limiting examples of ways in which a DCLTD may compromise lysosomal function include promoting lysosomal alkalinization, promoting lysosomal permeability, causing lysosomal release of cathepsins, inhibiting the ability of lysosomes to degrade proteins and lipids thus leading to their accumulation, prevention of fusion between lysosomes and autophagosomes and/or inhibition of autophagy. Such compromising of lysosomal function may occur as a consequence of disrupting a single critical regulator of lysosome function, such as control of lysosomal pH. Compromised lysosomal function may also be caused by a disruption of the ability of a cancer cell to compensate properly when exposed to agents that disrupt critical aspects of lysosomal regulation.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Thus, the discoveries of this invention enable an unprecedented co-ordinate regulation of a wide variety of genes and proteins critical in cancer biology, and that individually represent targets of potential therapeutic intervention. Only by the present invention, however, is it possible to target such a large number of these genes and proteins with a single therapeutic intervention.

In some selected embodiments, methods for restoring c-Cbl function or worsening lysosomal dysfunction in a patient in need thereof are provided. As used herein, the term “patient” refers to a human, companion animal such as a dog or cat, or livestock animal such as a horse, cow, pig, sheep and the like.

In some selected embodiments, c-Cbl function is restored by administering one or more agents that restores c-Cbl function. Such agents may be designated c-Cbl Restorative Agents (CRAs). Lysosomal dysfunction can be worsened by administering one or more agents that worsens lysosomal dysfunction. A single agent that both restores c-Cbl function (i.e., is a CRA) and worsens lysosomal dysfunction is designated a Dual c-Cbl Lysosomal Targeting Drug (DCLTD).

The restoration of c-Cbl function in a cancer cell can be assayed by analysis of increases in c-Cbl phosphorylation. In this approach, cancer cells are treated with a CRA or a DCLTD in the presence or absence of an agent that causes cells to become more oxidized. The status of c-Cbl phosphorylation may be analyzed by any of a number of means well-known to those skilled in the art, including Western blot analysis of cell lysates with an antibody that identifies phosphorylated c-Cbl, for instance at tyrosine residues Y731 or Y771, analysis of cell labeling with such antibodies which may be conjugated with a fluorescent probe that makes them suitable for analysis by fluorescence microscopy, immunohistochemistry or by automated fluorescence analysis of some form, or by CYTOF mass cytometry analysis. The change in phosphorylation status is compared with that seen in untreated cells, in cells treated with the CRA or DCLTD by itself, or that treated with a pro-oxidant by itself.

Alternatively, restoration of c-Cbl function can be determined by analysis of changes in the levels of direct targets of c-Cbl, with changes in the levels of two or more c-Cbl targets caused by the application of a single therapeutic intervention being an indicator of c-Cbl activation. In such analyses, cells are treated with saline, with the CRA or DCLTD by itself, with a pro-oxidant or with the combination of a CRA or a DCLTD with a pro-oxidant. The ability of treatment with the combined agents to cause greater decreases in the levels of proteins that are direct targets of c-Cbl provides a surrogate indicator of c-Cbl activation.

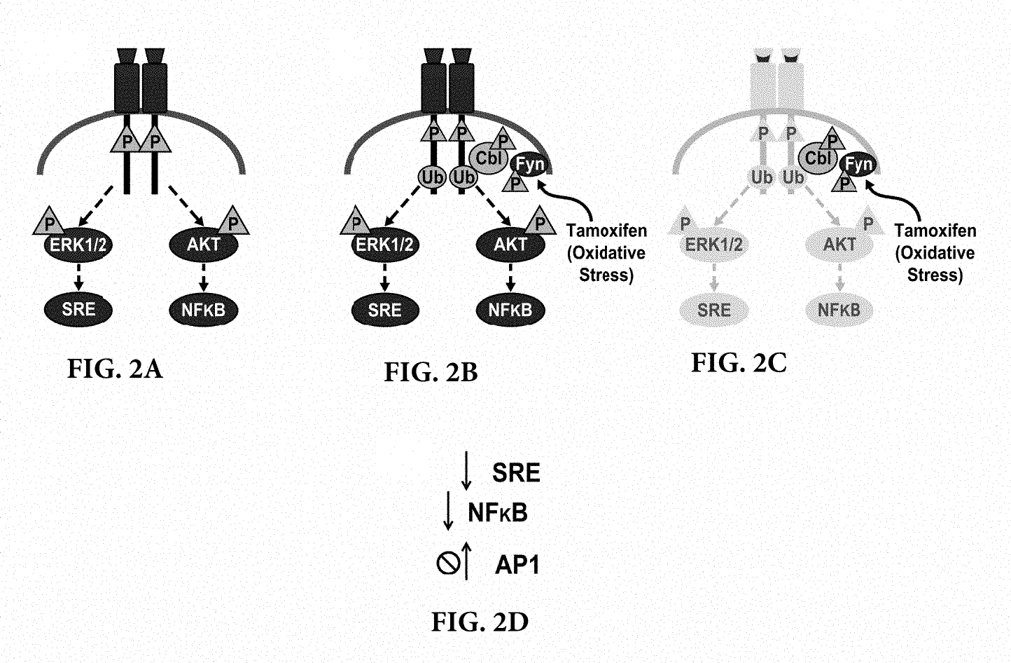

Alternatively, restoration c-Cbl function can be analyzed by changes in the level and/or activity of downstream targets of the direct targets of c-Cbl. Examples of this are provided in the drug screen provided in FIG. 2 that was used to identify the first examples of therapeutically useful CRAs and DCLTDs. In this screen, the activities of serum response element (SRE) and NF-κB in promoter-reporter essays was used to identify agents able to restore c-Cbl activation due to these transcriptional regulators having their activity downstream of multiple direct targets of c-Cbl. Multiple other downstream targets of the consequences of c-Cbl activation can equally be utilized in such analysis. For example, Notch-1, β-catenin and STAT5 (which are direct c-Cbl targets) all regulate multiple transcriptional targets that therefore are indirect targets of c-Cbl activation. Decreases in levels of Notch-1, β-catenin and/or STAT5 would lead to multiple predictable changes in downstream targets, the analysis of which would also provide surrogate indicators of c-Cbl activation. What is critical, however, in order to use such analysis for the analysis of c-Cbl activation is to analyze two or more targets that represent different downstream pathways affected as a consequence of c-Cbl activation and that are independently regulated, just as NF-κ and SRE are independently regulated.

Alternatively, analysis of c-Cbl activation can be conducted with surrogates chosen from the many genes whose regulation is altered as a consequence of treatment of cancer cells with CRA- or DCLTD-based therapies. Gene expression analysis is an important tool in studies of cellular function and is widely utilized, and its methodologies of analysis are well known to those practiced in the art. The identification of changes in gene expression patterns that are diagnostic of c-Cbl activation thus provides an additional means of examining the activity of a CRA- or DCLTD-based therapy. In some selected embodiments, the agent can increase c-Cbl function by at least 5%, 10%, 15%, 20%, 25%, 30% or more.

The worsening of lysosomal dysfunction can be assayed by multiple means known to those skilled in the art. Non-limiting examples of ways in compromising of normal lysosomal function can be detected include analysis of lysosomal pH, increases in lysosomal permeability, lysosomal release of cathepsins, decreases in the ability of lysosomes to degrade proteins and lipids thus leading to their accumulation, inhibition of fusion between lysosomes and autophagosomes and/or inhibition of autophagy. In some selected embodiments, the agent can increase lysosomal pH by at least 0.05, at least 0.1, at least 0.15, at least 0.2, at least 0.25, at least 0.3, at least 0.35, at least 0.40, at least 0.50, at least 0.75, at least 1.0 or at least 1.25 units. In some selected embodiments, the agent can increase cathepsin activation by at least 5%, at least 10%, at least 15%, at least 20%, at least 25%, at least 30%, at least 35%, at least 40%, at least 50%, at least 75%, at least 100%, or at least 125%. In some selected embodiments, the agent can increase phospholipid accumulation by at least 5%, at least 10%, at least 15%, at least 20%, at least 25%, at least 30%, at least 35%, at least 40%, at least 50%, at least 75%, at least 100%, or at least 125%.

Exemplary DLCTDs include, but are not limited to, estradiol propionate, neomycin, metoprolol, methylprednisolone, dequalinium, maprotiline, meclizine, naloxone, mechlorethamine, celastrol, carboplatin, acetyltryptophan, azadirachtin, proadifen, piracetam, cyclosporine, azacitidine, simvastatin, fluphenazine, monensin, mefloquine, perphenazine, sertraline, or mixtures thereof. DLCTD compounds having one or more ionizable functional groups (e.g., an amine, carboxylic acid and the like) may further be formulated as a pharmaceutically acceptable salt.

Provided herein are methods of increasing the sensitivity of cancer cells to one or more cancer therapies by restoring c-Cbl function, activating c-Cbl, and/or worsening lysosomal dysfunction. Increasing the sensitivity of cancer cells to cancer therapies expands the therapeutic options available to a cancer patient. For instance, restoring c-Cbl function, in the presence or absence of worsening lysosomal dysfunction, and preferably in combination with a compound that can be applied to a patient and that activates c-Cbl via the redox/Fyn/c-Cbl pathway, can increase the effectiveness of a particular therapeutic, or render an otherwise ineffective therapeutic effective.

In some embodiments, the present invention provides a method of treating cancer in a patient in need thereof, comprising: (a) identifying a patient with a cancer in which c-Cbl is inhibited, (b) administering one or more agents in an amount effective to restore c-Cbl function in the presence or absence of worsening lysosomal dysfunction, and preferably in combination with a compound that can be applied to a patient and that activates c-Cbl via the redox/Fyn/c-Cbl pathway, and (c) providing cancer therapy to the patient. In some embodiments, the agent (b) that restores the ability to activate c-Cbl comprises a DLCTD.

In some embodiments, the agent (a) comprises estradiol propionate, neomycin, metoprolol, methylprednisolone, dequalinium, maprotiline, meclizine, naloxone, mechlorethamine, celastrol, carboplatin, acetyltryptophan, azadirachtin, proadifen, piracetam, cyclosporine, azacitidine, simvastatin, fluphenazine, monensin, mefloquine, perphenazine, sertraline, mixtures thereof, or a pharmaceutically acceptable salt thereof.

In some embodiments, the cancer comprises melanoma, lymphoma, basal-like breast cancer, glioblastoma, pancreatic cancer, ovarian cancer, lung cancer, head and neck cancer, bladder cancer, colon cancer, or esophageal cancer cells.

In some selected embodiments, the cancer therapy includes any one of surgery, radiation therapy, chemotherapy, immunotherapy, hormone therapy, stem cell transplant or combinations thereof. In some preferred embodiments, the therapeutic regimen includes radiation therapy or chemotherapy, preferably chemotherapy.

In some embodiments, the cancer therapy comprises tamoxifen, carmustine, temozolomide, irradiation, or a combination thereof.

The chemotherapy can include a selective estrogen receptor antagonist such as lomifene, ormeloxifen, raloxifene, tamoxifen, toremifene, lasofoxifene, and ospemifene. A particularly preferred means of activating c-Cbl via the redox/Fyn/c-Cbl pathway is by combining the CRA or DCLTD with an agent such as tamoxifen due to its ability to provide estrogen receptor antagonistic effects along with estrogen receptor-α-independent benefits that include pro-oxidative activities. Tamoxifen is also a preferred agent to combine with a CRA or DCLTD as tamoxifen is also able to compromise lysosomal function. Thus, the combination of a CRA or a DCLTD with tamoxifen is a preferred illustration of the present invention, with tamoxifen also being replaceable with another agent that oxidizes cells, and that may also compromise lysosomal function

The chemotherapy can include alkylating agents, including (but not limited to) nitrogen mustards such as mechlorethamine (nitrogen mustard), chlorambucil, cyclophosphamide, ifosfamide, and melphalan, nitrosoureas such as streptozocin, carmustine (BCNU), and lomustine, alkyl sulfonates such as busulfan, triazines such as dacarbazine (DTIC) and temozolomide, and ethylenimines such as thiotepa and altretamine (hexamethylmelamine).

The chemotherapy can include a platinum drug, such as cisplatin, carboplatin, and oxalaplatin.

The chemotherapy can include an antimetabolite, such as 5-fluorouracil (5-FU), 6-mercaptopurine (6-MP), capecitabine, cytarabine, floxuridine, fludarabine, gemcitabine, hydroxyurea, methotrexate, and pemetrexed.

The therapies of this invention also can be used to enhance the therapeutic efficacy of multiple other treatments for cancer. For example, CRA- or DCLTD-based interventions, preferably applied in combination with an agent that activates c-Cbl via the redox/Fyn/c-Cbl pathway, will make cancer cells more susceptible to killing by any anticancer intervention by decreasing the expression and/or activity of multiple proteins that enhance cell survival and by decreasing the expression and/or activity of multiple proteins that are involved in therapeutic resistance. Such additional therapies may include, for example, exposure of cancer cells to tumor necrosis factor or derivatives thereof, to TRAIL, to inhibitors of receptors that promote cancer cell division and/or survival, immunotherapies and radiation.

In certain selected embodiments, the chemotherapy includes one or more agents that disrupt normal lysosomal function, or oxidize cancer cells. Many anticancer agents exhibit these properties, and sensitivity to them is therefore going to be amplified by application in combination with CRA- or DCLTD-based therapies. Examples of anticancer agents that have lysosomal disrupting properties and/or pro-oxidative properties include tamoxifen and carmustine, and buthionine sulfoximine. Anticancer agents also may have physicochemical characteristics that cause them to accumulate in lysosomes. Such agents also may have a free amine group that enables them to be protonated in the lysosome and thereby increase lysosomal pH, or they may inhibit lysosomal acidification and or re-acidification, may promote lysosomal permeabilization, may promote cathepsin release, may inhibit autophagy or any combination thereof. In some embodiments, the effectiveness of the cancer therapy can be enhanced by administering one or more agents intended to restore c-Cbl function and/or one or more agents that worsens lysosomal dysfunction prior to commencing treatment with a chemotherapeutic agent. In such pretreatment methods, cancer cells can be rendered more susceptible to cancer therapy. For instance, the duration and/or dosage of chemotherapy can be reduced. In certain embodiments, a patient can be treated with one or more agents intended to restore c-Cbl function, in the presence or absence of compromising lysosomal function and preferentially in conjunction with an agent that activates c-Cbl via the redox/Fyn/cCbl pathway, and/or one or more agents that worsens lysosomal dysfunction for a period of at least 1, 2, 3, 4, 5, 6, 7, 8, 9, 10 weeks prior to commencing the addition of an additional form of cancer therapy. In certain embodiments, the CRA or DCLT that is administered prior to commencing the therapeutic regimen, preferentially in combination with an intervention that increases c-Cbl activation via the redox/Fyn/c-Cbl pathway, is one that increases c-Cbl function by at least 5%, 10%, 15%, 20%, 25%, 30% or more. In certain embodiments, the agent that is administered prior to commencing the therapeutic regimen is one that worsens lysosomal dysfunction in combination with enabling activation of c-Cbl, for instance an agent that can increase lysosomal pH by at least 0.05, at least 0.1, at least 0.15, at least 0.2, at least 0.25, at least 0.3, at least 0.35, at least 0.40, at least 0.50, at least 0.75, at least 1.0 or at least 1.25 units. In some selected embodiments, the agent can increase cathepsin activation by at least 5%, at least 10%, at least 15%, at least 20%, at least 25%, at least 30%, at least 35%, at least 40%, at least 50%, at least 75%, at least 100%, or at least 125%. In some selected embodiments, the agent can increase phospholipid accumulation by at least 5%, at least 10%, at least 15%, at least 20%, at least 25%, at least 30%, at least 35%, at least 40%, at least 50%, at least 75%, at least 100%, or at least 125%.

In some embodiments, the effectiveness of the cancer therapy can be enhanced by administering one or more agents that are CRA or DCLTDs, in the presence or absence of one or more additional agents that worsens lysosomal dysfunction concurrently with the therapeutic regimen and preferentially in combination with an agent that increases c-Cbl activation via the redox/Fyn/c-Cbl pathway. In some embodiments, the effectiveness of the cancer therapy can be enhanced by administering one or more agents that are CRA or DCLTDs, in the presence or absence of one or more additional agents that worsens lysosomal dysfunction concurrently with the therapeutic regimen and preferentially in combination with an agent that increases c-Cbl activation via the redox/Fyn/c-Cbl pathway.

In some embodiments, the effectiveness of the cancer therapy can be enhanced by administering one or more CRAs or DLCTDs prior to, concurrently, or both prior to and concurrently with the therapeutic regimen. In certain embodiments, one or more additional CRA or DCLTDs, in the presence or absence of one or more additional agents that worsens lysosomal dysfunction concurrently with the therapeutic regimen and preferentially in combination with an agent that increases c-Cbl activation via the redox/Fyn/c-Cbl pathway can also be administered.

Increasing the effectiveness of the therapeutic can include a reduction in dosage levels of the therapeutic from what is conventionally employed by those of ordinary skill in the art. In some selected embodiments, increasing the effectiveness of the therapeutic can include a reduction of the period of time over which the therapeutic is conventionally administered. In certain embodiments, increasing the effectiveness of a therapeutic increases the potency and/or selectivity of the therapeutic against cancer cells and/or tumors.

Provided herein are methods of treating cancer by restoring c-Cbl function in the presence or absence of worsening lysosomal dysfunction. In some embodiments, the method includes identifying a patient that would benefit from the disclosed regimens by analyzing a cancer for reduced c-Cbl function and/or lysosomal dysfunction. The analysis may include one or more of pathological, histological or hematological assays. In some embodiments, the method includes identifying a patient that would benefit from the disclosed regimens by analyzing a cancer for reduced c-Cbl function. The analysis may include one or more of pathological, histological or assays of gene expression. For example, a biopsy may be analyzed to determine whether immune-precipitation of c-Cbl is associated with co-precipitation of a protein that is known to inhibit c-Cbl activity. As a non-limiting example of this approach, a biopsy of a brain tumor specimen may be analyzed by techniques known to those skilled in the art to determine whether or not there is a complex between c-Cbl and Cool-1/βpix. Alternatively, the tumor specimen can be assayed to determine whether Cool-1/βpix is phosphorylated, an activation state of the enzyme that is seen in tumor specimens but not in normal brain tissue. Such antibodies also can be directly applied in immunohistochemical analysis of tissue biopsy specimens. Such techniques may be applied to any protein that has been shown to bind to c-Cbl and inhibit its activity. Additionally, the analysis of gene expression patterns in tumors that are inhibited by c-Cbl by different means may reveal expression patterns indicative of particular inhibitory strategies employed for the cancer cell.

Evidence of decreased c-Cbl function may also be detected by observation of increases in protein levels of two or more direct targets of c-Cbl above that seen in normal body tissue, particularly in the absence of gene amplification or increases in mRNA expression for said c-Cbl targets. Additionally, evidence of inhibited c-Cbl function may be provided by analysis of gene expression patterns in tumor biopsy specimens. In cases in which inhibition of c-Cbl function is detected but the precise means by which c-Cbl is inhibited is not known, the patient may be treated with CRAs or DCLTDs, preferably in combination with an agent that activates c-Cbl via the redox/Fyn/c-Cbl pathway, known to be effective against different inhibitors in order to define a CRA or DCLTD that is more appropriate for that particular tumor. Alternatively, tumor cells derived from a particular patient may be analyzed in vitro for responsiveness to a battery of CRAs or DCLTDs, applied in the presence or absence of tamoxifen (or another agent that causes oxidation of cancer cells), in order to determine which CRA or DCLTD is most appropriate for use in that patient.

Having identified a patient that would benefit from CRA or DCLTD therapy, the patient can be given the optimal agent that restores c-Cbl function, as either a CRA or a DCLTD, based on the analysis above. In some embodiments, the methods include a step of selecting and administering the optimal agent that restores c-Cbl function, in the presence or absence of activity (in a DCLTD) or a separate agent worsens lysosomal dysfunction, based on the analysis above and applying said agent or agents in combination with a separate agent that oxidizes cells and thus activates c-Cbl via the redox/Fyn/c-Cbl pathway. The CRA/RFC pathway-based therapy may be applied in combination with additional agents, e.g., cancer chemotherapeutics, irradiation, cytokines that are able to kill tumor cells, immunotherapies or such other anticancer therapies know to those skilled in the arts.

In some selected embodiments, the cancer can include any one of clear cell sarcoma, breast, prostate, melanoma, pancreatic, ovarian, lung, head and neck, bladder, colon, esophageal, glioma, leukemia and lymphoma.

Provided herein are methods for treating cancer in a patient with a need thereof that include:
1) administering at least one agent (a) that is a compound that restores c-Cbl function, in the presence of absence of an additional agent to worsen lysosomal function worsens lysosomal dysfunction, or is a DLCTD;
2) administering at least one agent that can promote c-Cbl activation by oxidizing cells and thus activate c-Cbl via the redox/Fyn/c-Cbl pathway; and
3) administering at least agent (b) that is an anti-cancer agent.

In certain selected embodiments, the c-Cbl function and lysosomal condition of the cancer patient or tumor cells is evaluated prior to administering agent (a). The results of the evaluation enable one of ordinary skill in the art to select the optimal agents (a) and (b) for the individual patient. In some instances, the patient can be assessed for the likelihood that CRA or DLCTD therapy would improve the effectiveness of the anti-cancer agent. Such evaluation may be by analysis of immuno-precipitated c-Cbl from a tumor specimen to identify proteins with which it is complexed and/or by analysis of activation status of c-Cbl and determining levels of c-Cbl targets and/or by analysis of proteins that inhibit c-Cbl for diagnostic changes in, e.g., their phosphorylation status and/or by analysis of gene expression profiles to detect changes in gene expression indicative of c-Cbl inhibition.

In some embodiments are provided methods for treating cancer in a patient with a need thereof that include:

1) administering at least one agent (a) that is a compound that restores c-Cbl function (i.e., a CRA) in the presence or absence of a compound that compromises lysosomal function, or is a DLCTD; and/or

2) administering (a) in combination with (b) TMX or another agent that oxidizes cells and thus can activate c-Cbl via the redox/Fyn/c-Cbl pathway and/or

3) administering (a and b) in combination with at least one standard anti-cancer agent.

In certain selected embodiments, the c-Cbl function of the cancer patient or tumor cells is evaluated prior to administering agent (a). Analysis of c-Cbl function may be carried out by direct analysis of c-Cbl itself to examine its phosphorylation status, or its presence in a complex with inhibitory proteins of by determination of levels of c-Cbl phosphorylation in tumor cells by any of the methods of immunological analyses well known to those skilled in the art. Analysis of c-Cbl function also may be carried out by examining levels of expression of c-Cbl targets. If multiple known c-Cbl targets are elevated in the levels of their protein expression above that seen in normal tissue, particularly in the absence of associated gene amplification of these targets, then it is most likely that the cancer represents one in which c-Cbl is inhibited. The results of the evaluation enable one of ordinary skill in the art to select the optimal agents (a) and (b) for the individual patient.

For instance, a patient with cancer has a tumor biopsy removed, which may be for the purposes of pathological examination and/or for the purposes of surgical reduction of the tumor mass. A portion of this tissue is delivered as fresh tissue to an analytical laboratory, such as a pathology laboratory. Alternatively, the tissue may be frozen and then delivered to the analytical site as frozen tissue. The tissue is then lysed and c-Cbl is immuno-precipitated, for example with beads coated with anti-c-Cbl antibody, or by other means known to those skilled in the art. The material that has been immuno-precipitated is then analyzed for the presence of inhibitory proteins that may bind to c-Cbl. One means of conducting such analysis is by probing polyacrylamide gels on which the sample has been run with antibodies against Cool-1/βpix, transglutaminase-2, Sprouty2, CIN85 apoptosis-linked gene 2-interacting protein X (Alix), STS1, STS2 or other proteins that will be discovered that are able to bind to c-Cbl and inhibit its normal function.

Another means of analyzing the immuno-precipitated protein complex is by analysis using mass spectrometry in order to identify those proteins that have co-immunoprecipitated with c-Cbl. Identification of the inhibitory protein or proteins to which c-Cbl is bound then enables selection of an appropriate CRA or DCLTD to utilize in the therapies of the present invention. In some instances, in this example, tissue may also be analyzed with antibodies directed against a protein that binds directly with c-Cbl or at that activates a pathway that causes inhibition of c-Cbl. An example of a protein that binds directly with c-Cbl is Cool-1/βPix, while an example of a protein that activates a pathway that causes inhibition of c-Cbl is Cdc42. Such antibodies would necessarily be directed against an epitope or epitopes that indicate activation of said protein in such a manner that inhibition of c-Cbl is likely. An example of such an epitope is the phospho-serine(340) epitope that is recognized on Cool-1/βpix by the commercially available antibody anti-phospho-serine(340) antibody sold by Millipore.

In some instances, antibodies directed against an epitope or epitopes indicative of the existence of a complex of c-Cbl with an inhibitory protein may be used to directly examine tissue in immuno-visualization assays. Such assays may be, for example, immunofluorescence or immuno-peroxidase assays. In such examinations, the tissue is incubated with the primary antibody directed against an epitope or epitopes expressed by a protein that may either inhibit c-Cbl directly or may activate a pathway that leads to inhibition of c-Cbl. Using standard criteria for identifying cells as cancer cells, the binding of the primary antibody will then be visualized. This can be accomplished either by using an antibody that itself is labeled to enable visual detection or by incubating the cells or tissue with a second antibody able to bind to the first antibody but that also is conjugated to a chemical that enables visualization by, for example, fluorescence visualization or enzymatic amplification reactions. An example of the latter category would be an immuno-peroxidase reaction.

Such assays also can be used to examine tumor cells that are extracted from the bloodstream as individual cells or are extracted from ascites fluid as individual cells. Such cells would be fixed in order to enable visualization of binding to antigens inside of the cell or, if present in sufficient number to enable biochemical analysis, could be examined in the same manner as tissue biopsies. If cell numbers are limited, then immuno-visualization techniques of the type used to examine tissue biopsies can also be applied with cells followed by their examination by any type of microscopic or automated cell analysis technologies, such as a fluorescence activated cell sorter. Such labeling can be conducted in conjunction with other markers, for example markers of cancer stem cells, in order to define subpopulations of cancer cells that are most likely to provide evidence of inhibition of c-Cbl function.

Expression of such epitopes is considered in comparison with normal (non-cancerous) tissue in order to determine whether expression levels are different in the cancer cells.

Analysis of Cool-1/βpix phosphorylation can be conducted by any of several different manners well known to those skilled in the arts. In one example of such an analysis, an antibody directed against a specific phosphorylated amino acid on Cool-1/βpix that is indicative of its activation and that is associated with the presence of a complex between c-Cbl and Cool-1/βpix is used in a Western blot analysis to indicate the presence of Cool-1/βpix that is phosphorylated at that specific site at increased levels as compared with normal (non-tumorous) tissue. Another example of a method by which such analysis can be conducted is that Cool-1/βpix is immuno-precipitated from a tissue biopsy or from cancer cells by incubating cells with, for example, beads that are labeled with anti-Cool-1/βpix antibody. After the immuno-precipitation procedure and appropriate washing steps, the Cool-1/βpix is now removed from the beads and is analyzed by Western blot analysis using antibodies directed against phospho-tyrosine or phospho-serine in order to determine the phosphorylation status of the Cool-1/βpix. Alternatively, the immuno-precipitated Cool-1/βpix may be analyzed directly by mass spectrometry in order to provide a map of phosphorylated amino acids on the protein.

An example of an antibody that can be used as a demonstration of the general principle of this prophetic example is the Millipore antibody directed against the phospho-serine(340) residue of Cool-1/βpix. As presented in multiple points within the description of this invention, this antibody may be used to detect phospho-Cool-1/βpix by Western blot analysis or by immuno-visualization analysis.

One means of determining whether c-Cbl inhibition is likely to be present within a tumor sample is to analyze the levels of protein expression of multiple c-Cbl targets, with a minimum of two targets but preferably of four or more targets being analyzed. If multiple c-Cbl targets are elevated in their expression, compared with levels of expression in normal tissue controls, then tissue or cells will be analyzed to examine whether increases in protein levels are associated with gene amplification and/or increases in mRNA expression for the c-Cbl targets. The range of targets that may be examined in this assay is large, and continues to grow, with the only restriction being that the proteins examine should be ones that are known to be overexpressed in the particular type of tumor being examined. For example, an examination of many tumors would include an examination of levels of the epidermal growth factor receptor while a much more restricted set of tumors, and predominantly lymphoid tumors, would be examined for the expression levels of Flt3, a c-Cbl target that is predominantly overexpressed in tumors of the hematopoietic system. Examples of targets of c-Cbl that are candidates for analysis in the above assays include, as non-limiting examples, FLICE-inhibitory protein (c-FLIP), epidermal growth factor receptor, ephrin type-A receptor 2, ErbB2, fibroblast growth factor receptor-2, FLt3, gp130, insulin-like growth factor-I receptor, the c-MET receptor for hepatocyte growth factor; platelet-derived growth factor receptor-α; PI3 signal transducer and activator of transcription 5.

Analysis of gene expression patterns in tumor biopsy samples, or in isolated tumor cells, also may be used to determine the presence of c-Cbl inhibition. For example, activation of c-Cbl in glioblastomas is associated with the decreased expression of mRNA for several hundred different genes that appear to be coordinately regulated by c-Cbl activation. Increased levels of such genes, or of a subset of such genes consisting of 10 more members of the gene set controlled by c-Cbl activation, can be used to provide a further indication of the presence of c-Cbl inhibition.

Analysis of gene expression patterns also may be used to identify specific means of c-Cbl inhibition. For example, analysis of glioblastoma specimens reveals that approximately 50% of such specimens show increased protein levels of two or more different proteins that are direct targets of c-Cbl and also show increased levels of mRNA for Cool-1/βpix.

If there was a combined expression of the gene expression patterns indicative of c-Cbl inhibition in combination with increased levels of a protein known to be capable of inhibiting c-Cbl function, this would provide another means of identifying which c-Cbl inhibitor, or c-Cbl inhibitors, is/are relevant to that cancer cell. Such information would indicate the appropriate CRAs/DCLTDs to use in treatment of cancer in that patient.

In tumors in which evidence is provided that c-Cbl function is inhibited by any of the means discussed above, and the inhibitor is one for which appropriate CRAs/DCLTDs do not yet exist, then the tumor cells can be utilized to identify new CRAs/DCLTDs using precisely the same approaches as defined above. Such CRAs/DCLTDs would then be used according to the steps of the present invention.

In some embodiments, agents (a) and (b) are administered in separate compositions.

In certain selected embodiments, agents (a) and (b) are administered in a single composition. Accordingly, one aspect of the invention includes pharmaceutical compositions containing (a) and (b).

The pharmaceutical compositions containing agents (a) and (b) can be prepared according to known methods, for example by means of conventional dissolving, lyophilising, mixing, granulating or confectioning processes.

Solutions and suspensions of agents (a) and (b), for instance isotonic aqueous solutions or suspensions can be used. By way of example, agents (a) and (b) can be lyophilized, optionally in combination with various carriers and excipients. The lyophilized compositions can be reconstituted in water or other liquid prior to administering to a patient. Oils for suspensions include vegetable, synthetic or semi-synthetic oils customary for injection purposes.

Pharmaceutical compositions for oral administration can be obtained by combining agents (a) and (b) with solid carriers and/or other excipients, and processing into tablets, capsules and the like. The agents (a) and (b) can further be formulated into dosage forms exhibiting controlled, extended or enteric release. In some selected embodiments, it is preferred that the agent (a) is formulated for immediate release, while agent (b) is formulated for controlled, extended or enteric release.

Also provided herein are methods for treating a resistant cancer. Chemotherapy resistance in cancer cells is often regulated by c-Cbl ubiquitin ligase. In some embodiments, the methods disclosed herein include methods of treating a resistant cancer comprising administering one or more agents that restore c-Cbl function to a patient in need thereof. In some instances, the cancer is resistant to tamoxifen (TMX).

The number of means by which cancer cells can acquire resistance to TMX is very large, with over 40 different mechanisms implicated thus far in acquired TMX resistance. Resistance mechanisms include changes in expression of ERα and/or changes in ERα-related signaling, but the great majority of such mechanisms are independent of ERα signaling. These include increased expression of receptor tyrosine kinases (RTKs) that promote division and/or survival, increased expression of proteins that block apoptosis, constitutive activation of pro-survival signaling pathways, decreased expression of pathways (such as transforming growth factor-β signaling) that suppress tumor growth and increased (or decreased) expression of specific miRNAs. Thus, overcoming acquired TMX resistance requires significant attention to effects of this agent independent of ERα.

Many of the TMX resistance mechanisms that do not involve changes in ERα expression and/or signaling also are known to confer resistance to multiple other anticancer agents. Thus, expression of these resistance pathways may render cells refractory to other therapeutic approaches.

A further problematic means by which cells become resistant to TMX is through expression of characteristics of tumor initiating cells (TICs, also referred to as cancer stem cells). Indeed, growth of cancer cells in the presence of TMX or other anticancer agents has been reported to select for cells with properties of TICs. As one of the characteristics of TICs is to be resistant to a large variety of anticancer therapies, including both irradiation and pharmacological agents, elimination of cells with TIC properties can be particularly difficult. Increases in TIC frequency also may contribute to increased malignancy and tendency to metastasize.

The existence of so many diverse means by which cells become TMX resistant raises the possibility that targeting any single mechanism will simply select for cells that utilize a different strategy to escape treatment; thus, an important strategy for overcoming resistance is likely to require simultaneously targeting of multiple resistance mechanisms. This approach would be analogous to that used for treating such diseases as tuberculosis or AIDS.

One of the intriguing aspects of the resistance mechanisms for TMX or other anticancer agents that have been discovered thus far is that many of these mechanisms share a common feature of being regulated by c-Cbl. For example, c-Cbl activation causes downregulation of vascular endothelial growth factor receptor-2 (VEGFR2), HER2, RON, the insulin-like growth factor-I receptor (IGF-IR), Eph A2 and the epidermal growth factor receptor (EGFR). Enhanced degradation of these receptor tyrosine kinases (RTKs) leads to decreased activity of their downstream targets, including PI3 kinase (which is also a c-Cbl target), Akt, Erk1/2 and such further downstream cellular regulators as AIB1, mTOR, and NF-kB. Activated c-Cbl also targets activated β-catenin (an effector of Wnt signaling, which also is implicated in chemoresistance. These examples provide a non-limiting indication of the potential importance of c-Cbl as a controller of therapeutic resistance.

Also provided herein are methods of killing cancer stem cells by administering a CRA in the presence or absence of an agent that worsens lysosomal function, in the preferred presence of an agent that oxidizes cells and thus is able to activate c-Cbl via the redox/Fyn/c-Cbl pathway. In some embodiments, the methods include a step of identifying the optimal agent that restores c-Cbl function, worsens lysosomal dysfunction, or both. The agent that restores c-Cbl function is preferentially combined with an agent that oxidizes cells and thus activates c-Cbl via the redox/Fyn/c-Cbl pathway. The agent(s) can be additionally combined with one or more anti-cancer agents to kill cancer stem cells.

This invention also provides methods of eliminating cancer cells from bone marrow by treatment with a CRA, in the presence or absence of an agent that worsens lysosomal function, or with a DCLTD, in preferred combination with an agent that oxidizes cells and thus can activate c-Cbl via the redox/Fyn/c-Cbl pathway. Said treatment may be combined with one or more standard anti-cancer agents. In some embodiments, the methods include the step of harvesting bone marrow cells from a mammal and treating the harvested bone marrow cells with at least one agent that is a CRA, in the presence or absence of an agent that worsens lysosomal function, or with a DCLTD, in preferred combination with an agent that oxidizes cells and thus can activate c-Cbl via the redox/Fyn/c-Cbl pathway. A preferred oxidizing agent is tamoxifen. The method can further include the step of treating the harvested bone marrow cells with at least one anti-cancer agent. Exemplary anti-cancer agents include, but are not limited to, cyclophosphamide, busulfan, mephalan, temozolomide, cisplatin, BCNU or multiple other agents known to those skilled in the arts. In some selected embodiment, the bone marrow is treated with an agent that is a CRA, in the presence or absence of an agent that worsens lysosomal function, or with a DCLTD, in preferred combination with an agent that oxidizes cells and thus can activate c-Cbl via the redox/Fyn/c-Cbl pathway prior to the anti-cancer agent, is treated concurrently with the anti-cancer agent, or it treated with the agent both prior to and concurrently with the anti-cancer agent. After the stem cells have been purged, the bone marrow may be transplanted back to the mammal.

Provided herein also is a triangulating drug screen that enables rapid identification of agents that restore c-Cbl function in cancer cells. Activation of c-Cbl leads to accelerated degradation of receptor tyrosine kinases that are c-Cbl targets, which leads to decreased activation of ERKs1/2 and Akt. Two transcriptional regulators that are downstream of these signaling proteins are the serum response element (SRE) and NF-κB, respectively. Thus, if activation of c-Cbl is achieved by a particular drug, then there will be decreased activation of both SRE and NF-κB. In this way, there is a rapid way to eliminate from further consideration compounds that are inhibitors of SRE or NF-κB themselves. However, a screen for compounds that cause decreases in the activity of both SRE and NF-κB has the drawback, in that similar results would be obtained by agents that generally decreased gene transcription, whether due to being transcriptional inhibitors or through other regulatory pathways. The method thus provides a third screen for function of the transcriptional regulator AP-1: compounds that restore c-Cbl function either have no effect on AP-1 activity, or increase AP-1 activity. This screen can be further enhanced by addition of other screens representing pathways that also are downstream of c-Cbl activation but that can be regulated separately from receptor tyrosine kinases that are c-Cbl targets. Examples of such additional screens can include screens for activity of Notch-1, β-catenin, STAT5 or other transcriptional regulators that are themselves regulated by c-Cbl.

One of the challenges in drug discovery is that often when an assay is conducted to find a drug that affects a particular enzyme, receptor, transcription factor other biochemical entity, the assay does not provide sufficient information as to the precise point in the regulatory architecture of the cell at which the drug is working. For example, there has been a great deal of interest in cancer research in finding inhibitors to the transcription factor NF-κB, and multiple chemical substances have been described that cause decreases in activity of this transcription factor. However, the assays conducted do not provide information on whether the decreased activity is due to inhibition of NF-κB itself or to inhibition of activities that are upstream or downstream of the transcription factor, or that are otherwise required for its activity (such as binding partners, as one example). Moreover, it may be that it is necessary to discover means of regulating a biological activity in the absence of knowledge as to a specific biochemical entity that represents a preferred drug target. Similarly, many screens for anticancer agents look at the effects of, for example, single agents on cell survival or proliferation. Only rarely are screens designed to look at more than one outcome on a particular cell type in such a manner as to define a novel drug target, and screens designed to use analysis of multiple outcomes to identify compounds that target a precise point in a pathway for which no known pharmacological agents exist do not appear in studies on drug identification. Despite these problems inherent in standard drug screening approaches that rely on single outcomes to determine whether a compound is of sufficient interest for further examination, they remain the mainstay of drug discovery research. In addition, even when more than one outcome is examined, screens often are not conducted in a manner that provides more accurate identification of the point at which a drug may act in order for a desired outcome to be achieved.

The triangulating drug screen provided illustrated in FIG. 2 offers a non-limiting example of an effective and novel assay for identifying agents with the desired activity of restoring c-Cbl function by combining two or more outcomes that enable triangulation on the activity of interest. Following the principles of radio triangulation, triangulating drug screen assays work by first identifying at least two separate and measureable activities that represent divergent outcomes of activating or inhibiting the enzyme or transcription factor or receptor(s) of interest. These outcomes must represent independent outcomes. As such, these activities cannot represent a linear signaling outcome such that activity 2 depends upon activity 1 as such a linear outcome would not provide two independent vectors. Instead, triangulating drug assays are carried out by employing activities that are dependent upon the protein/activity of interest but are not dependent upon each other. In a preferred embodiment of this triangulating drug discovery assay, a third activity outcome is included that eliminates spurious outcomes that may influence assay outcome in ways that do not reflect the activity for which the screen is being conducted. Still additional activities may be added to further narrow the range of possible means of achieving the desired outcomes

In a non-limiting example of a triangulating drug screen a novel method is provided for discovering drugs that restore the ability to activate c-Cbl in cancer cells. These agents are called c-Cbl Restorative Agents, or CRAs. As proof of principle of the ability of this screen to identify novel drug activities, screening was carried out on a library of 1040 compounds consisting of compounds that have been extensively studied and the majority of which are already approved by the FDA or other regulatory agencies for use in humans. None of these compounds are known to have the desired activity of restoring function of c-Cbl in cancer cells, which is an activity that has not previously been recognized for any of the agents identified or for any of the therapeutically useful agents contained within any of the compound libraries available from MicroSource Discovery or in other comparable libraries.

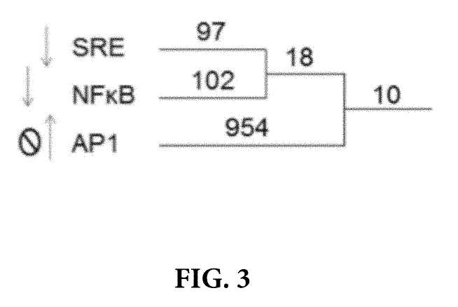

The triangulating drug screen that was designed to identify agents that restore the function of c-Cbl is illustrated in FIG. 3. The challenge faced in designing this assay was that drug discovery assays for finding agents able to restore c-Cbl function in cancer cells are inherently difficult to conceive. Assays of the activity of purified c-Cbl would be of uncertain, if any, relevance, as the necessary goal is to restore c-Cbl function in situations in which such function is suppressed. The function of c-Cbl is to modify proteins by attaching ubiquitin to them, but there are multiple enzymes that engage in ubiquitylation and thus this known outcome of activating c-Cbl does not provide information that necessarily identifies c-Cbl itself. The screen that was designed to identify CRAs that would restore c-Cbl function when it is suppressed in cancer cells provides a functional example of a triangulating screen.

As the key means of achieving therapeutic benefit in the present invention is to activate c-Cbl via a molecular mechanism that does not restrict its activity to a single one of its targets (and thus in this manner is distinguished qualitatively from canonical means of c-Cbl regulation), it follows that any mechanism that enables such a broad activation of c-Cbl will be to all intents and purposes identical in its outcomes to that achieved by activation of c-Cbl via the redox//Fyn/c-Cbl pathway. For example, Fyn kinase may be activated by multiple means, such as activation as a consequence of integrin activation or activation by other biochemical pathways in which Fyn kinase is thought to play an important role. In any circumstance in which Fyn kinase is activated, its ability to activate c-Cbl would have the same effects as described when the kinase is activated due to oxidation-induced activation. As another example in which c-Cbl may be activated to achieve the same outcomes as activation via the redox//Fyn/c-Cbl pathway, Fyn kinase is a member of the Src family of kinases and other members of this family also may be activated by oxidation and also have been reported as being able to activate c-Cbl. Thus, Fyn kinase is not viewed as an exclusive means of activating c-Cbl to achieve the purposes of the present invention and activation via other enzymes that are able to activate c-Cbl would be similarly effective and would fall within the domains of the present invention. For example, such members of the src family of kinases may be Lyn kinase or Yes kinase, and it is readily appreciated that there may be cancer cells in which it is possible to exploit for therapeutic purposes a redox/Lyn/c-Cbl pathway or a redox/Yes/c-Cbl pathway, as non-limiting examples of this invention. All of these different means of activating c-Cbl would fall within the invention of combining activation of c-Cbl with a metabolic change that activates an enzyme that is an intermediary between the metabolic change and the activation of c-Cbl.

Disclosed herein are pharmaceutical compositions including (a) at least one agent that restores c-Cbl function, at least one agent (b) that oxidizes cancer cells thereby activating c-Cbl via the redox/Fyn/c-Cbl pathway and (c) a chemotherapeutic. Also disclosed herein are methods for identifying a CRA agent using the triangulating drug screen described herein, including of determining the activity of at least two proteins that are altered in their activity by activation of c-Cbl.

### EXAMPLES

The following examples are for the purpose of illustration of the invention only and are not intended to limit the scope of the present invention in any manner whatsoever.

### Example 1: Identification of c-Cbl Restorative Agents

To identify candidate agents able to restore c-Cbl function, we designed a three-vector screen based on our findings that activation of c-Cbl in oligodendrocyte progenitor cells (OPCs) caused receptor tyrosine kinase (RTK) degradation, decreases in Erk1/2 and Akt activation and decreased transcriptional activity by NF-kB (which is downstream of Akt activation by RTKs) and by the serum response element (SRE, which is downstream of Erk1/2 activation by RTKs) (FIG. 2). We used standard promoter-reporter constructs to identify compounds that caused decreases in NF-κB and SRE activity in GBM cells, based on the deduction that simultaneous decreases in both outcomes would most likely be caused by something upstream of both transcription factors, such as RTK activity. However, such an outcome also could be caused by any agent that suppressed gene transcription. Therefore, to eliminate general transcriptional inhibitors, we restricted our interest to compounds that had no effect on, or elevated, AP-1 activity. This now provided a screen in which the three outcomes are all independent of each other, but can only occur simultaneously in a limited number of ways.

The screen that was designed to identify CRAs that would restore c-Cbl function when it is suppressed in cancer cells provides a functional example of a triangulating screen. To identify candidate agents able to restore c-Cbl function, we designed a three-vector screen based on our findings that activation of c-Cbl in oligodendrocyte progenitor cells (OPCs) caused receptor tyrosine kinase (RTK) degradation, decreases in Erk1/2 and Akt activation and decreased transcriptional activity by NF-kB (which is downstream of Akt activation by RTKs) and by the serum response element (SRE, which is downstream of Erk1/2 activation by RTKs). We used standard promoter-reporter constructs to identify compounds that caused decreases in NF-κB and SRE activity in GBM cells, based on the deduction that simultaneous decreases in both outcomes would most likely be caused by something upstream of both transcription factors, such as RTK activity. Moreover, to eliminate general transcriptional inhibitors, we restricted our interest to compounds that had no effect on, or elevated, AP-1 activity. This now provided a screen in which the three outcomes are all independent of each other, but can only occur simultaneously in a limited number of ways.

This screen identifies a unique constellation of outcomes that eliminates, in this case, compounds that only inhibit NFκB or serum response element promoter activity, and requires that both outcomes are decreased.

This screen further took advantage of the fact that the luciferase enzyme used in reporter-promoter assays is itself an oxidase, and thus oxidizes cells in which it is expressed. Thus, expression of luciferase is able to promote activation of c-Cbl via the redox/Fyn/c-Cbl pathway if the inhibition of c-Cbl is overcome. However, this approach can also be implemented by adding to the cancer cell cultures a pro-oxidizing agent.

The potency of the triangulating screen approach is demonstrated by the outcomes. In a screen on 1040 compounds assayed at concentrations of 0.1, 1 and 5 μM. 102 compounds were identified that decreased NF-kB activity and another 97 that decreased SRE activity. Yet only 18 compounds had both effects. Moreover, only 10 of the 18 compounds either elevated or had no effect on AP-1 activity. These final 10 compounds (Table 1) are structurally diverse and were not previously known to have any common properties (and include a tetracyclic antidepressant, an estro-mimetic, two antibiotics, a β-1 receptor blocker, a steroid derivative, an anti-malarial, an opiate receptor antagonist, an anticancer agent and an anti-histamine).

TABLE 1

| Drug | Major drug function |
|---|---|
| Estradiol Propionate | estromimetic |
| Neomycin Sulfate | Aminoglycoside antibiotic |
| Metoprolol Tartrate | β1 receptor blocker |
| Methylprednisolone | Steroid derivative |
| Dequalinium Chloride | Antiseptic, disinfectant |
| Maprotiline Hydrochloride | Tetracyclic antidepressant |
| Meclizine Hydrochloride | Antihistamine, antiemetic |
| Naloxone Hydrochloride | opiate receptor antagonist |
| Mechlorethamine | Alkylating agent |
| Celastrol | Tetracyclic triperneoid |

Six of the 10 compounds remaining were chosen to determine whether they restored the ability of oxidation of cells to cause decreases in levels of a c-Cbl target, an outcome that occurs when c-Cbl is activated via the redox/Fyn/c-Cbl pathway. These compounds indeed restored the ability of pro-oxidative stimulation to cause decreases in the levels of the epidermal growth factor receptor (EGFR), which is a c-Cbl target known to be important in growth of many cancer cells and that is of interest as a potential target in cancer treatment.

Other examples of downstream consequences can be used to create triangulating drug screens for the discovery of agents that restore c-Cbl function in cancer cells. As a non-limiting example, Notch-1, β-catenin and STAT5 are all transcriptional regulators that are targeted by c-Cbl for degradation. Thus, these targets could be substituted for or combined with the use of NF-κB and/or SRE as reporter outcome measures. If a screen is conducted that utilizes more than two promoters that are directly or indirectly downstream of c-Cbl activation, then decreasing the activity of any other transcriptional regulator that is a direct target of c-Cbl or that is itself regulated (directly or indirectly) of a direct target of c-Cbl can be used an outcome measure. Moreover, a screen outcome does not necessarily have to represent a decrease in activity. For example, if degradation of a c-Cbl target caused increased activity of a downstream reporter of any sort (which could be a transcriptional regulator, an enzymatic activity, a cellular function or a metabolic state), then this too could be incorporated into the triangulating drug screen to identify agents that restore c-Cbl function. Non-limiting examples of genes that increase in their expression following restoration of c-Cbl function, and that would offer possible outcomes in which expression and/or activity is increased as a result of c-Cbl activation. Although the screen described was used to identify CRAs, the principles embodied in this screen has multiple other applications that will be obvious to those skilled in the art and can be applied in any situation in which it is possible to generate multiple independent readouts that collectively identify a specific pathway, protein, gene or metabolic state.

In the specific examples provided herein, the ability of compounds of interest to worsen lysosomal dysfunction were initially determined in two ways. In some cases, compounds identified in this group had previously been reported to have the ability to potentially alter lysosomal function. Second, other members of this group of drugs were revealed in a screen in which OPCs were exposed to a concentration of galactosylsphingosine (GalSph, which is also referred to as psychosine) that kills 50% of OPCs. We found that GalSph disrupts multiple lysosomal functions, including lysosomal pH, accumulation of neutral and phospholipids and endo-lysosomal trafficking. Analysis of the same library of 1040 compounds used to identify compounds able to restore function of c-Cbl revealed >200 compounds that greatly increased cell death when applied to cells exposed to levels of GalSph that caused death of only 50% of cells. The list of compounds that were able to exacerbate lysosomal toxicity of GalSph contained every drug revealed in our first screen as able to restore activation of c-Cbl.

It is important to stress that having the ability to worsen lysosomal dysfunction is not sufficient in and of itself to restore c-Cbl function as the great majority (>90%) of the compounds that exacerbated the toxicity of GalSph had no effect on decreasing levels of activation of NF-κB or SRE, and thus were not candidates for restoring the ability to activate c-Cbl.

As a further example of the general principles of applying a triangulating drug screen to the discovery of pharmacological agents that restore the ability to activate c-Cbl in cancer cells, a separate screen was conducted using the MDA-MB-231 basal-like breast cancer (BLBC) cell line. We previously showed that c-Cbl activation is inhibited in these cells by Cdc42, and thus differed from glioblastoma cells in which c-Cbl is inhibited by Cool-1/β-pix. Cdc42 Knockdown had analogous effects in BLBC cells as Cool-1/β-pix knockdown in GBM cells (with experiments conducted in seven different cell lines representing three major types of BLBCs), thus demonstrating the biological importance of c-Cbl inhibition was similar to that seen for GBM.

As predicted if the specific mechanism of c-Cbl inhibition is predictive of therapeutic agent specificity, conducting the identical drug screen described in FIG. 2 on BLBC cells yielded different compounds than the GBM screen (Table 2). We identified 12 structurally and functionally diverse compounds that decreased transcription by NF-κB and SRE, but not by AP-1 in MDA-MB-231 cells, but none of these compounds were the ones revealed in the GBM cell screen. The drugs revealed in these two independent screens are shown in Table 2. Thus, as a demonstration that drugs identified in the screens conducted are highly specific in their effects, the drugs that were identified as effective on GBM cells were not detected in the screen on BLBC cells, and vice versa. As these two types of cancer cells inhibit c-Cbl by different mechanisms, the outcomes demonstrate that the drugs identified are dependent on the mechanism by which c-Cbl is inhibited and further demonstrate the ability of the screen to identify a distinct set of drugs with the desired properties.

Surprisingly, all of the candidate CRAs discovered in analysis of MDA-MB-231 cells also shared the characteristic of the drugs in Table 1 to compromise lysosomal function, thus confirming these drugs also as DCLTDs. As for the drugs discovered in the GBM screen, some of the drugs identified had previously been reported to have the ability to potentially alter lysosomal function. Second, other members of this group of drugs were revealed in the screen in which OPCs were exposed to a concentration of GalSph that killed only 50% of cells. In the list of >200 compounds that greatly increased cell death when applied to cells exposed to levels of GalSph that caused death of only 50% of cells, all compounds found in the screen on MDA-MB-231 cells were present. Also as with the screen on GBM cells, having the ability to compromise lysosomal function is not sufficient in and of itself to restore c-Cbl function as the great majority (>90%) of the compounds that exacerbated the toxicity of GalSph had no effect on decreasing levels of activation of NF-κB or SRE, and thus were not candidates for restoring the ability to activate c-Cbl.

TABLE 2

| Cell line examined | Drug |
|---|---|
| Glioblastoma | Estradiol Propionate |
| Glioblastoma | Neomycin Sulfate |
| Glioblastoma | Metoprolol Tartrate |
| Glioblastoma | Methylprednisolone |
| Glioblastoma | Dequalinium Chloride |
| Glioblastoma | Maprotiline Hydrochloride |
| Glioblastoma | Meclizine Hydrochloride |
| Glioblastoma | Naloxone Hydrochloride |
| Glioblastoma | Mechlorethamine |
| Glioblastoma | Celastrol |
| Basal-like breast cancer | Carboplatin |
| Basal-like breast cancer | Acetyltryptophan |
| Basal-like breast cancer | Azadirachtin |
| Basal-like breast cancer | Proadifen Hydrochloride |
| Basal-like breast cancer | Piracetam |
| Basal-like breast cancer | Cyclosporine |
| Basal-like breast cancer | Azacitidine |
| Basal-like breast cancer | Simvastatin |
| Basal-like breast cancer | Fluphenazine Hydrochloride |
| Basal-like breast cancer | Monensin Sodium |
| Basal-like breast cancer | Mefloquine |
| Basal-like breast cancer | Perphenazine |
| Basal-like breast cancer | Sertraline Hydrochloride |

### Example 2

One of the possible consequences of restoring normal c-Cbl activation in a cancer cell in which such activation is inhibited is that this will increase the sensitivity of cancer cells to estrogen receptor-α (ERα)-independent activities of tamoxifen (TMX).

To increase c-Cbl activation, the application of a CRA or DCLTD-based therapy designed to activate c-Cbl via the RFC pathway needs to overcome the great variability within cells of a tumor, or between tumors, in oxidative status. Different cancer cells may have different intracellular redox states, thus leading to differing levels of c-Cbl activation via the RFC pathway even when this opportunity for c-Cbl activation is restored. Cancer cells in different locations (in different patients or within a single patient) also may be exposed to differing degrees of pro-oxidative signals (such as tumor necrosis factor-α) thus providing different levels of oxidation and potentially different levels of c-Cbl activation via the RFC pathway. In order to create a therapeutically useful level of activation in different cancer cells, it is preferable to combine restoration of c-Cbl function, preferably with a DCLTD, with exposure to an additional agent that causes oxidation of the cancer cells. In the example provided, this is accomplished by combining the CRA or DCLTD with TMX as a non-limiting example of the benefits of such a combination. Tamoxifen is applied at the low microM concentrations that generally are utilized to benefit from its ERα-independent activities. TMX results provide a non-limiting example, however, of this approach as many anti-cancer agents (including irradiation) are known to oxidize cells and thus would also be able to activate c-Cbl via the RFC pathway. In a non-limiting example of this general principle, the DCLTD maprotiline (MPT) was combined with TMX and glioblastoma cells were treated with these agents. MPT is a tetracyclic antidepressant identified in the screens of Example 1. MPT enters the central nervous system (CNS) and can be used safely for long periods of time. It has only rarely been studied as an anticancer agent, and there are no indications it has ever been studied in combination with TMX. In the example provided, the intent is to activate c-Cbl via the redox/Fyn/c-Cbl pathway, in this case utilizing TMX's activities as a pro-oxidant. Although best known for its activity as an estrogen receptor-α (ERα) antagonist, clinically relevant low μM TMX concentrations have multiple ERα-independent effects of potential therapeutic relevance, including pro-oxidative activity. These ERα-independent effects have led to clinical studies on the use of high dose TMX in treatment of GBMs, other gliomas, and a dozen other types of cancers.

Of particular importance, and adding to the novelty of the invention of this application, although the ERα independent activities of TMX have been of such broad interest that this agent been studied in clinical trials in over a dozen different cancers, there has been no rational approach to harnessing TMX's ERα-independent activities in order to make it more effective. Indeed, there is little evidence that it provides any benefit in these situations, which is a predicted outcome if c-Cbl were inhibited in these cancers. The present invention provides a solution to this problem and harnesses TMX's pro-oxidative activities to enhance c-Cbl activation via the RFC pathway and also harnesses TMX's lysosome disrupting properties to further build on the ability of MPT to exacerbate mild lysosomal dysfunction.

In prior studies on attempts to apply the ERα-independent activities of TMX in cancer treatment, there is no rational exploitation of the activities of TMX as a pro-oxidant. Moreover, there are no previous studies that rationally exploit TMX's pro-oxidant activities in cancer treatment, other than our own studies on the combination of TMX with an experimental Cdc42 inhibitor (called ML141) that is not clinically useful and that only provided partial suppression of tumor growth that was not sufficient to be of clinical value. Thus, the idea that treatment of tumors with a pharmacological agent able to restore c-Cbl function might provide any therapeutically relevant outcomes is not just not anticipated by prior studies but in fact is counter-indicated by our studies on ML141 in which suppression of tumor growth was only partial.

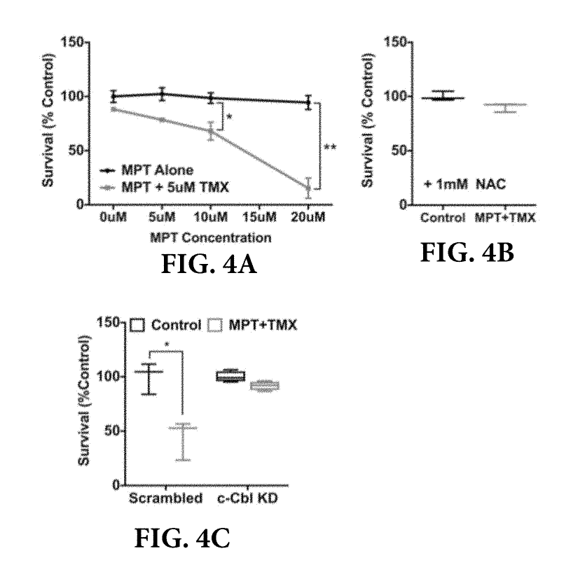

Exposure of glioblastoma cells to clinically relevant concentrations of MPT enhanced the sensitivity of GBM cells to concentrations of TMX that are clinically relevant in high dosage TMX usage (FIG. 4). This outcome was observed, for example, in five different glioblastoma cell lines isolated from different patients. The cell lines studied represent a range of glioblastoma subtypes and genotypes and generate human glioblastomas when transplanted into the CNS of immune-deficient mice.

As several studies have suggested that tryicyclic antidepressants (TCAs) might also be useful in treatment of malignant gliomas, the effects of several TCAs previously studied by others also were examined. Despite a rich representation of TCAs in the NINDS-II library, none of these compounds caused decreases in both NF-κB and SRE activity in GBM cells. In addition, examination of the ability of fluoxetine, amitryptiline and imipramine to increase the sensitivity of GBM cells to TMX revealed that none of them were as effective as MPT in this regard, even though these agents were identified as potentially able to compromise lysosomal function by previous studies in the literature and/or by their enhancement of GluSph toxicity. Thus MPT, the DCLTD revealed in the triangulating drug screen that provides a method for discovering this and other CRAs and DCLTDs, has/have novel properties in respect to effects on GBM cells and these activities are not expressed by other antidepressants of a different structural class. These above results reinforce the distinctive nature of the compounds that are able to restore c-Cbl function and the unique nature of DCLTDs. This distinctive nature of the DCLTDs is reinforced by the use of TMX itself. Several studies have shown that one of the ERα-independent effects of TMX is to alter lysosomal function and even to make lysosomes more alkaline, yet TMX exposure by itself is not sufficient to restore normal c-Cbl regulation. Such observations further reinforce the unique nature of DCLTDs and demonstrate that compromising of lysosomal function is not sufficient for an agent to be a CRA.

The properties of TMX as a lysosomal disruptor also suggest that it is a particularly suitable partner for combination with a DCLTD, by both making cells more oxidized and by contributing to further disruption of normal lysosomal function. Both of these properties are of great interest in terms of developing a combined therapy with a DCLTD, and may be used in identifying particularly rational combinatorial therapies that include a DCLTD. Pharmacological restoration of c-Cbl function, even with an agent such as MPT (which causes decreases in levels of Cool-1/β-pix, as shown in FIG. 9), would not be expected to have the similar effects as genetic knockdown of a target protein. In the case of pharmacological agents that function as direct inhibitors, the fact that they are generally not irreversible generally renders them less effective than genetic knockdown of a protein. Even in the case of an agent that, like MPT, can cause decreases in levels of the inhibitory protein, such decreases are unlikely to be as complete as obtained by genetic knockdown.

### Example 5

As a further demonstration of the general applicability of the principles of this invention, another drug identified in the screen on GBM cells, meclizine, also increased the vulnerabilty of GBM cells to TMX. Meclizine is an anti-histamine used as an anti-emetic and has not been previously identified as able to increase the sensitivity of cancer cells to TMX or to have any activities of potential relevance to the restoration of c-Cbl function in cancer cells.

### Example 6

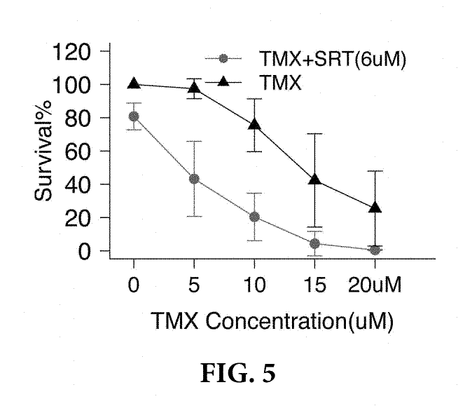

As another demonstration of the general applicability of the principles of this invention, the drug sertraline (which was identified in the screen on MDA-MB-231 cells), increased the sensitivity of these cells to TMX (FIG. 5). In addition, mefloquine, a second drug identified as a DCLTD in screens on MDA-MB-231 cells, also increased the sensitivity of melanoma cells to TMX. Sertraline and mefloquine both were identified in screens on MDA-MB-231 BLBC cells, in which c-Cbl is inhibited in a Cdc42-dependent manner.

### Example 7

A further example of the potency of therapies that restore c-Cbl function, and of DCLTDs in particular, is provided by the circumstances in which cancer cells utilize more than one inhibitor of c-Cbl in order to suppress function of this enzyme. For example, in some circumstances it appears that Cool-1/βpix and Cdc42 are interactive partners and that this interaction is required for their function. Although this does not appear to be the case for GBM or BLBC cells analyzed thus far, it nonetheless is a possibility. In such circumstances, the cells would be sensitive to drugs identified as a result of the screen of GBM cells or of the screen of BLBC cells.

One example of the possible sensitivity of cancer cells to agents that restore function when c-Cbl when more than one inhibitor is utilized by that cancer cell is provided by studies on melanoma cells. MPT also increased the sensitivity of melanoma cells to TMX. Moreover, a second drug identified as a DCLTD, mefloquine, also increased the sensitivity of melanoma cells to TMX. This may represent a cancer cell in which inhibition of c-Cbl is mediated by both Cdc42 or Cool-1/βpix, either working independently or in partnership, and where inhibition of either pathway is sufficient to restore sufficient c-Cbl function in order to obtain the benefits of this therapeutic approach.

### Example 8

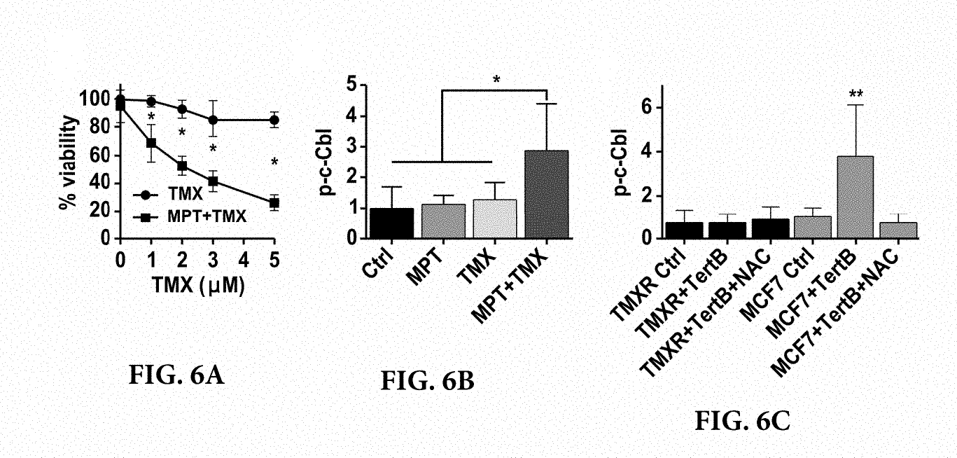

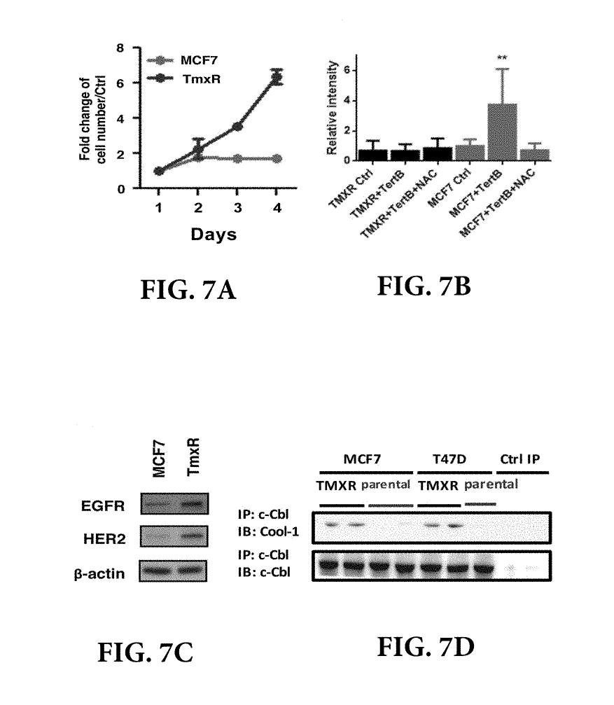

A further example of the broad relevance of the principles of this invention is provided by the findings that DCLTD-based treatments may be used to overcome acquired TMX resistance (FIG. 6). Treatment of luminal breast cancer (LBC) with TMX, or other hormonal modifying agents, is one of the great success stories of modern oncology but nonetheless is associated with the emergence of resistance to treatment in a significant proportion of patients. Treatment of TMX-resistant LBCs is an enormous challenge, in part because resistance can occur by many (>40) different means. Resistance mechanisms include changes in expression of ERα and/or changes in ERα-related signaling, but the great majority of such mechanisms are independent of ERα signaling. These include increased expression of receptor tyrosine kinases (RTKs) that promote division and/or survival, increased expression of proteins that block apoptosis, constitutive activation of pro-survival signaling pathways, decreased expression of pathways (such as transforming growth factor-β signaling) that suppress tumor growth and increased (or decreased) expression of specific miRNAs. Thus, overcoming acquired TMX resistance requires significant attention to effects of this agent independent of ERα.

The existence of so many diverse means by which cells become TMX resistant raises the possibility that targeting any single mechanism will simply select for cells that utilize a different strategy to escape treatment; thus, an important strategy for overcoming resistance is likely to require simultaneously targeting of multiple resistance mechanisms.

In examining TMX resistance mechanisms that have been discovered thus far, we made the novel observation that many of these mechanisms share a common feature of being regulated by the c-Cbl E3 ubiquitin ligase, either directly or indirectly (as indicated by various examples provided in the present invention). For example, c-Cbl activation causes downregulation of vascular endothelial growth factor receptor-2 (VEGFR2), HER2, RON, the insulin-like growth factor-I receptor (IGF-IR), Eph A2 and the epidermal growth factor receptor (EGFR). Enhanced degradation of these receptor tyrosine kinases (RTKs) leads to decreased activity of their downstream targets, including PI3 kinase (which is also a c-Cbl target), Akt, Erk1/2 and such further downstream cellular regulators as AIB1, mTOR, and NF-kB. Activated c-Cbl also targets activated β-catenin (an effector of Wnt signaling). All of these indicated proteins may contribute to TMX resistance, and the discoveries of the present invention thus provide a potential strategy to take on the entirely novel goal of attacking multiple resistance mechanisms with a single therapeutic intervention.

We used standard techniques of generating TMX-resistant (TMX-R) MCF-7 cells by growing them for 6 months in the presence of 1 μM TMX. Unlike their parental MCF-7 cells, the TMX-R cells continued to divide when grown in the presence of 1 μM TMX (a dose that is cytostatic but not lethal for the MCF-7 population used in these experiments). TMX-R cells showed increased expression of EGFR and HER2, both of which are c-Cbl targets (FIG. 3B). Cool-1 knockdown caused decreases in levels of HER2 and EGFR. While Cool-1 was expressed in both parental and TMX-R cells (which is not surprising given its putative role in cytoskeletal regulation. Cool-1 knockdown increased c-Cbl phosphorylation and greatly reduced the ability of TMX-R cells to grow as mammospheres. Perhaps most striking were observations that Cool-1 knockdown greatly decreased the ability of TMX-resistant cells to generate tumors. It was also of interest that TMX-R cells showed no loss of ERα (not shown), in agreement with many other studies indicating that acquired TMX resistance occurs for reasons other than loss of this receptor.

### Example 9

An example of the ability of DCLTDs to restore the ability of TMX to enhance phosphorylation of c-Cbl via the redox/Fyn/c-Cbl pathway is shown in FIG. 8. Co-treatment of glioblastoma cells with MPT enabled TMX-induced c-Cbl phosphorylation. As predicted if activation was RFC pathway-mediated, such activation was inhibited by N-acetyl-L-cysteine (NAC, a glutathione pro-drug and anti-oxidant), thus confirming that TMX-induced activation of c-Cbl was dependent on the pro-oxidative activities of TMX. The fact that TMX alone was not sufficient to activate c-Cbl demonstrated the necessity of combining this pro-oxidative activity with a CRA or a DCLTD.

### Example 10

The ability of MPT to restore c-Cbl function is unprecedented, and could have many explanations (such as inhibiting activation of Cool-1, targeting other unknown c-inhibitors in the affected cells, or directly affecting the inhibitory complex). Understanding how this occurs is an important component of understanding the biology of DCLTD-based therapies.

Studies on how MPT restores c-Cbl function provide an illustrative example of a potential mechanism of action of a CRA, or of a CRA that is also a DCLTD. MPT treatment of glioblastoma cells decreases levels of the inhibitory protein Cool-1/βpix. This example also provides the first evidence for the existence of compounds, and particularly of therapeutically relevant compounds, that cause decreases in levels of proteins that are able to inhibit c-Cbl function and thus reveals a new biological activity and therapeutically relevant target activity. This example also provides the first evidence for a pharmacological agent able to decrease levels of Cool-1/βpix and also to greatly decrease the complexing of Cool-1/βpix with c-Cbl. These analyses demonstrate that exposure of GBM cells to MPT decreases the complex between c-Cbl and Cool-1/β-pix and causes decreases in levels of Cool-1/β-pix. Moreover, even though decreases in levels of Cool-1/βpix were not complete, MPT exposure appeared to cause a virtually complete elimination of complexes between c-Cbl and Cool-1/β-pix (FIG. 8). Not only are there no prior instances of therapeutically useful agents that are able to restore the normal regulation of c-Cbl function in cancer cells, there also are no agents that are known to affect the inhibition of c-Cbl by Cool-1/β-pix or to decrease complex formation between c-Cbl and Cool-1/βpix. There also are no agents that are known to decrease levels of Cool-1/β-pix. This treatment appears to be the first example of a pharmacological intervention that restores c-Cbl function in cancer cells in which its normal function is compromised by an inhibitory protein, and the first example of a pharmacological intervention that decreases levels of the inhibitory protein.

### Example 11

A further example of the general applicability of the approach of the present invention is provided by studies on TMX-resistant luminal breast cancer cells. Treating of these cells with maprotiline also causes a decrease in levels of the complex between c-Cbl and Cool-1/βpix (FIG. 9).

### Example 12

The many years of research on cancer biology have identified a large number of proteins that are thought to offer potential therapeutic targets of value in controlling cancer cell growth. Efforts by many investigators, in academic laboratories and companies, have focused on the analysis of inhibitors of many of these potential targets. Examples of such inhibitors include the inhibitors of specific receptors (such as the epidermal growth factor receptor, HER2, c-Met and many others), of specific transcription factors (such as NF-κB, FoxM1 and many others), of proteins that promote cell survival (such as c-FLIP, Bcl-2 and many others) and of intermediate signaling proteins (such as Akt, PI3 kinase, mTOR and many others). Some of these efforts have been successful enough to lead to the generation of clinically utilized products, but they all suffer from the shortcomings that they are of relatively little use in the successful targeting of malignant tumors and that cancer cells have many mechanisms for escaping the targeting of a single one of these regulatory nodes.

Clearly, what would be a far preferable as a therapeutic approach is one that enables the targeting of multiple of these proteins with a single therapeutic intervention. Prior to this invention, however, no such approach existed. Although there is much discussion about identifying key “hubs” of control in cancer cells (i.e., proteins that control multiple functions), and it has proven possible to identify a small number of such hubs (e.g., the p53 protein), targeting of such hubs for therapeutic purposes has thus far generally not been effective.

One theoretical intervention that might enable targeting of multiple proteins important in cancer cell function would be the ability to activate c-Cbl to degrade multiple of its targets at the same time. The vast majority of studies on c-Cbl function provide no means of accomplishing this, as the canonical means of activating c-Cbl is related to its interaction with individual targets. For example, the canonical path to c-Cbl activation is exemplified by the activation of the EGFR. This leads to phosphorylation and activation of multiple proteins, one of which is the c-Cbl ubiquitin ligase. Activation of c-Cbl causes it to attach ubiquitin to the EGFR, thus targeting it for degradation. In this way, c-Cbl functions as a negative regulator of receptor tyrosine kinase signaling.

Thus, although it has long been recognized that it is possible to prevent c-Cbl activation in a manner that interferes with its ability to cause degradation of its multiple target proteins (primarily by mutation of c-Cbl, but more recently also by expression of proteins that inhibit c-Cbl activation), there has been no method of activating c-Cbl in such a manner as to promote degradation of multiple target proteins with a single therapeutic intervention.

The identification of the redox/Fyn/c-Cbl pathway provides a potential opportunity to activate c-Cbl in such a manner that its activation was not tied to a single receptor. Studies on the effects of genetic knockdown of Cool-1/β-pix in GBM cells indicated that such an effect might be able to be harnessed as this knockdown caused decreases in levels of the three c-Cbl targets of Notch-1, β-catenin and EGFR.

Treatment with MPT+TMX caused decreases in levels of the multiple c-Cbl targets, thus demonstrating that this pharmacological approach is able to cause decreases in levels of multiple c-Cbl targets with a single therapeutic intervention. Due to the activation of c-Cbl via the RFC pathway, it is predicted that multiple other direct targets of c-Cbl will be found to be decreased in their levels.

As illustrated in FIG. 10, treatment of GBM cells with MPT+TMX caused decreases in levels of the c-Cbl targets of EGFR, Notch-1 and β-catenin, as well as platelet-derived growth factor receptor-α (PDGFRα). As predicted by the redox/Fyn/c-Cbl hypothesis of c-Cbl activation, decreases were less marked when cells were exposed to MPT on its own. Moreover, these decreases were blocked by c-Cbl knockdown, demonstrating the dependence of the observed effects on the ability to activate c-Cbl.

### Example 13

A further demonstration of the ability of DCLTD-based therapies to cause decreases in levels of direct and indirect c-Cbl targets is provided by treatment of the MA2 melanoma cell line. Treatment with MPT+TMX caused decreases in levels of HSP70 and β-catenin, and similar outcomes were obtained by treatment with mefloquine+TMX. The similar results obtained by utilizing either MPT or mefloquine is in agreement with the ability of both of these agents to increase the sensitivity of melanoma cells to TMX and further supports the interpretation that melanoma cell inhibition of c-Cbl may be mediated by both Cool-1/βpix and Cdc42 and that intervention at either point will provide benefits of therapeutic relevance.

### Example 14

Treatment with MPT+TMX also caused decreases in levels and/or activation of other proteins that are thought to be critical in cancer cell function but which are not direct targets of c-Cbl (FIG. 11). There are two groups of such proteins, ones in which the connection to a c-Cbl target can be reasonably surmised based on prior research and ones for which the linkage to c-Cbl is currently unknown. Non-limiting examples of the first category of outcomes are activities of Erk1/2 and Akt, which both are downstream of receptor tyrosine kinases that are targets of c-Cbl. Further non-limiting examples are activity levels of NF-κB and serum response element (SRE), which are activated downstream of Akt and SRE, respectively. We also found that treatment with MPT+TMX caused decreases in levels of FoxM1, Sox2, heat-shock factor 1 (HSF-1), and heat shock protein 70 (HSP70), which are not thought to be c-Cbl targets and for which the linkage to targets of c-Cbl is currently unknown. Such an outcome is both unprecedented and of great scientific and therapeutic interest, as research efforts have been established to find inhibitors of each of these single proteins, but there is no prior demonstration of any intervention that enables targeting of the group of them with a single therapeutic intervention. These proteins are not direct targets of c-Cbl, and some of them (such as FoxM1 and Sox2) do not yet have established regulatory pathways that link them back to direct targets of c-Cbl or indirect targets of c-Cbl.

The observation that exposure to TMX on its own does not have the above effects underscores the importance of combining a pro-oxidizing agent with an agent that restores the ability to activate c-Cbl. Such an outcome is as predicted from the biological principles underlying this invention, because the pro-oxidizing effects of tamoxifen are predicted to be limited in cancer cells in which the ability to activate c-Cbl via the RFC pathway is compromised.

Another example of the unprecedented potency of the present invention to attack multiple proteins important in cancer cell biology with a single therapeutic intervention is provided by analysis of mRNA levels of critical cancer control proteins. To achieve control at this level of cellular analysis is unprecedented, in particular as c-Cbl is studied as a regulator of degradation of existing proteins rather than as a regulator of gene expression.

Quantitative PCR (qPCR) analyses of genes encoding proteins critical in cancer cell regulation show c-Cbl dependent decreases in transcription of FoxM1, Sox2 and HSP70 (FIG. 11).

Still other cancer regulatory proteins that thus far are decreased in their mRNA expression (as determined by global deep sequencing analysis of mRNA expression) by exposure of GBM cells to MPT+TMX include targets ID3, WNT3, Sprouty2, Sox9, Myc binding protein (MycBP), PI3 kinase regulatory subunit 3. Treatment with MPT+TMX also caused increases in the putative tumor suppressors FoxO3, FoxO4, and Sirtuin 2. All of these genes/proteins are of interest as potential therapeutic targets for treatment of GBM and other cancers (e.g., [35, 72, 82, 136-181]).

The number of transcriptional regulators that now have been identified to be decreased in levels and/or activity as a consequence of treatment with a DCLTD+TMX is sufficiently large as to indicate that this treatment will cause a significant changes in still larger patterns of gene expression in GBM cells. Analysis of these changes using methods known to those skilled in the relevant arts will provide a pattern of gene expression that can be used to determine if a tumor is characterized by inhibition of c-Cbl.

Thus, the discoveries of this invention enable an unprecedented co-ordinate regulation of a wide variety of genes and proteins critical in cancer biology, and that individually represent targets of potential therapeutic intervention. Only by the present invention, however, is it possible to target such a large number of these genes and proteins with a single therapeutic intervention.

### Example 15

As multiple studies have shown the potential value of targeting each of the individual proteins decreased in their levels by restoration of c-Cbl function, it is important to consider whether directly targeting any of these proteins has similar effects as CRA-based therapies. Thus far there is no evidence that targeting these individual proteins has the same effects as treatment with a DCLTD+a pro-oxidant, emphasizing the unique nature of CRA/DCLTD-based therapies. For example, exposure of cells to concentrations of thiostrepton reported to inhibit FoxM1 does not increase sensitivity to TMX as potently as does MPT and is not associated with a notable increase in c-Cbl phosphorylation. The HSP70 inhibitor PUH7 is still less effective than thiostrepton at increasing TMX sensitivity.

Thus, it currently seems most likely that the beneficial effects of c-Cbl restoration are not singularly dependent on any individual protein whose levels are decreased in association with c-Cbl activation. Moreover, the decreases in multiple potential therapeutic targets that are caused by CRA/DCLTD-based treatments represent partial inhibition of multiple therapeutic targets rather than standard approaches aiming at near-complete inhibition of single targets. The CRA/DCLTD-based therapies thus offer a new strategy of causing partial inhibition of multiple proteins important in cancer cell function with the single therapeutic intervention of a [CRA/DCLTD+a pro-oxidant (e.g., TMX)]. This enables a new approach to cancer therapy that is distinct from attempts to cause total inhibition of individual cancer control proteins.

### Example 16

It is increasingly recognized that cancers contain a subset of cells that are particularly important in tumor generation. These cells are called tumor-initiating cells (TICs, which also are referred to as cancer stem cells). Elimination of TICs is one of the central goals of ongoing research on new cancer therapies. These cells represent a subset of cells in a cancer, but are the cells that are able to readily generate a tumor when transplanted. A great deal of research has been directed at finding ways to eliminate TICs, as the failure to eliminate cells with TIC function is thought to lead with high probability to tumor recurrence. Identification of TICs by antigen expression or other phenotypic properties has, however, been incompletely effective in that cells that do not express putative phenotypic markers of TICs may also be able to generate tumors.

While the precise identity of TICs in different tumors may be controversial, it is generally agreed that cancer stem cells are more resistant to cancer treatments, such as chemotherapy and irradiation, than are the other cells that comprise the tumor. This provides a particularly difficult therapeutic challenge, as it may be possible to cause marked reductions in tumor volume with a particular therapy, but if the TICs are not eliminated then the cancer will inevitably recur. Due to this central importance of cancer stem cells in tumor biology, there have been many efforts to identify cancer stem cells and to develop means of eliminating them.

One of the great challenges in eliminating TICs is that the molecular mechanisms that are important in maintaining TIC biology are also critical in the biology of normal stem and progenitor cells. Thus, the question of how to attack TICs without causing unacceptable levels of damage to normal tissue represents a difficult and critical challenge in the development of new cancer treatments.

One of the promising outcomes of genetic restoration of c-Cbl function by Cool-1/β-pix knockdown in GBM cells or knockdown of Cdc42 in BLBC cells is virtual elimination of TICs. However, both of these approaches result in complete or near-complete elimination of these c-Cbl inhibitors and it is unlikely that the pharmacological approaches that will be necessary to develop therapeutically relevant interventions will be similarly effective. Indeed, the extent of decrease in levels of Cool-1/βpix caused by exposure to MPT was less than that caused by expression of shRNAi in GBM cells.

Unexpectedly, in light of the partial reduction in levels of Cool-1/βpix, and the similarly partial reduction in levels of proteins important in TIC biology, treatment with DCLTD-based therapies eliminates TICs. For example, GBM cells were exposed to sublethal combinatorial concentrations of 10 μM MPT+1 μM TMX for 5 days in vitro, drug concentrations that kill about ˜10% of total cells (FIG. 12). After treatment, 500,000 live cells were transplanted intra-cranially in NSG mice. Cells treated with saline or with temozolomide (the first-line treatment for glioblastoma) generated tumors within three weeks, as detected by photon emission from luciferase-expressing cancer cells, while cells treated with MPT+TMX showed no tumor generation even after 5 months. Thus, treatment with temozolomide had little apparent effect on TIC elimination, in agreement with patient data indicating rapid recurrence of tumor even during the period of temozolomide treatment.

Exposure of GBM cells to the combination of MPT+TMX led to the apparently complete elimination of GBM cancer stem cells. GBM cells from four different cell lines were exposed to MPT+TMX combination for 72 hrs in vitro, after which 500,000 live cells were transplanted intra-cranially in NSG mice. Cells treated with vehicle alone or with tamoxifen all generated tumors within three weeks, while cells treated with MPT+TMX showed no tumor generation even after 5 months.

Similar effects were seen on tumor spheroid generation in vitro even at sublethal doses of [MPT+TMX], providing an in vitro assay for analysis of other candidate therapeutic agents.

### Example 17

A further example of the novel features of DCLTD-based therapies is that these therapies are more lethal for TICs than for non-TICs, but are able nonetheless to eliminate both cellular populations at clinically relevant exposure levels. At the same time, these therapies do not increase the vulnerability of primary oligodendrocyte precursor cells to anticancer agents. One of the most challenging aspects of developing effective cancer treatments is that most anticancer treatments have the problem of being able to cause great levels of toxicity to the normal cells of the body. Thus, it is a desirable (but rarely achieved) feature of any new cancer treatment to be able to increase toxicity for cancer cells without causing at the same time increases in toxicity for normal cells. This is a particularly difficult problem to solve in the context of cells of the central nervous system, as many of these cells are inherently more sensitive to anticancer agents than are cancer cells themselves.

Previous studies have shown that one of the cell types of the brain that is most sensitive to the adverse effects of multiple chemotherapeutic agents are the oligodendrocyte progenitor cells (OPCs) that give rise to the myelin-forming oligodendrocytes of the central nervous system. Due to the high degree of sensitivity of OPCs to anticancer treatments, they provide a preferred cell type for use in evaluating whether new treatments exhibit desirable levels of specificity for cancer cells.

Analyses conducted on OPCs isolated from the developing human central nervous system demonstrated that MPT did not increase the sensitivity of these progenitor cells to tamoxifen (FIG. 13).

This outcome is not solely the property of MPT. FIG. 13B shows that treatment with sertraline (a CRA/DCLTD identified in studies on basal-like cancer cells rather than glioblastoma cells) also does not increase sensitivity of OPCs to TMX.

The ability to selectively increase the toxicity of TMX in cancer cells and not in vulnerable primary cells is a general property of CRAs/BLBCs. As shown in FIG. 13C, the experimental Cdc42 inhibitor CASIN also does not increase the vulnerability of OPCs to TMX. This is so even though CASIN does not provide the apparent protective activity that appears to be provided by sertraline, indicating that the selective effects of CRA/DCLTD treatment represent an important new general principle.

Even more important, the selective effects of treatment with CRAs/DCLTDs are not limited to TMX. FIG. 13D demonstrates that treatment with MPT, and even treatment with MPT+TMX does not increase the sensitivity of OPCs to temozolomide (TMZ, the front-line treatment for glioblastoma). It also does not increase the sensitivity of OPCs to 4-hydroxycyclophosphamide, a chemotherapeutic agent used in the treatment of multiple different cancers, including breast cancers.

### Example 18

What is particularly novel about the therapies provided in the present invention is that they actually are suitable for transition from the laboratory to clinical studies, particularly as the present examples of these therapies are focused on the discovery of new properties and methods of use of compounds that already are approved for use in humans.

As discussed earlier, ERα-independent effects of TMX have been of sufficient interest have led to clinical studies on the use of high dose TMX in treatment of GBMs, other gliomas, and a dozen other types of cancers. In all of these studies, however, there is no rational exploitation of the activities of TMX as a pro-oxidant. Moreover, none of the outcomes in these prior studies have been sufficiently compelling as to make the use of TMX of anything other than experimental interest thus far. Such a lack of useful outcomes is as predicted if c-Cbl activity is inhibited in tumor cells, as we would predict to be the case at least for glioblastoma cells, basal-like breast cancer cells, and luminal breast cancer cells with acquired tamoxifen resistance.

The only previous studies that rationally exploit TMX's pro-oxidant activities in cancer treatment are our own studies on the combination of TMX with an experimental Cdc42 inhibitor (called ML141) that is not clinically useful and that only provided partial suppression of tumor growth that was not sufficient to be of clinical value. Thus, the idea that treatment of tumors with a pharmacological agent able to restore c-Cbl function might provide any therapeutically relevant outcomes is not just not anticipated by prior studies but in fact is counter-indicated by our studies on ML141 in which suppression of tumor growth was only partial.

As an example of the unexpected potency of CRA/DLCTD-based therapies, treatment with a DCLTD (maprotiline)+TMX+TMZ was used to treat of established GBMs growing intracranially in immune-deficient mice. Immune-deficient NOD-Scid-gamma (NSG) mice were transplanted intra-cranially (1 mm to the left of the midline, 0.5 mm anterior to coronal suture and 3 mm deep) with 500,000 luciferase-expressing human GBM cells derived from three different GBM patients. In all experiments, tumors were allowed to grow until they were clearly visible by non-invasive imaging with an Advanced Molecular Imager (AMI; Spectral Instruments), which generally took two-three weeks. Mice were then assigned to different treatment groups, with a distribution that ensured equal representation of different tumor sizes in each group. Mice were treated by oral gavage with saline, TMZ, or MPT+TMX+TMZ, as all drugs in this group are orally available. Mice were treated, in a regimen of 5 days on/2 days off, until all saline-treated mice were dead (at 9 weeks after treatment initiation). All drugs were applied at clinically relevant exposure levels.

TMZ treatment transiently suppressed tumor growth, but re-initiation of rapid tumor growth was apparent while treatment was ongoing (as often occurs in patients); in contrast, treatment with MPT+TMX+TMZ showed complete suppression of tumor growth throughout the time of treatment (FIG. 14). For example, for one GBM line (identified as GBM27 tumors (“27” in FIG. 14), tumor size (as determined by photon flux) in TMZ-treated animals did not increase until the 4^{th} week of treatment (when tumor size had already increased ˜100-fold in saline treated mice), but at the 9-week (end of treatment) time point, size had increased >100-fold (p<0.01). In contrast, tumor size in mice treated with [MPT+TMX+TMZ] was less than 3-fold greater at 9 weeks than at the beginning of treatment. At 12 weeks post-treatment, tumor size in TMZ-treated mice was >5000-fold greater than at the start of treatment, but there was only a 9-fold increase in tumor size in mice treated with [MPT+TMX+TMZ]. Results with GBM10 cells (“10” in FIG. 14) were similar, except that here the tumor size 3 weeks after cessation of [MPT+TMX+TMZ] treatment was <0.3% the starting size, as compared with >500-fold increases in TMZ treatment.

### Example 19

The most important test of the potential utility of DCLTD/RFC pathway-based treatments is the ability to control the growth of established tumors in vivo. Analysis of the utility of DCLTDs as a potential cancer treatment was first focused on MPT for several reasons. First, glioblastomas (GBMs), the most malignant of gliomas and other brain tumors, represent one of the greatest challenges in all of cancer treatment. GBMs exhibit resistance to chemotherapeutic agents, irradiation and other cell death inducers. GBM cells can colonize brain tissue far removed from the tumor's primary origin, thus creating a tumor that is essentially metastatic in the brain and effectively eliminating the ability to achieve a significant therapeutic benefit by surgically removing the main tumor mass. Moreover, tumors may be located in parts of the brain and spinal cord for which surgery is extremely dangerous. Glioblastomas also exhibit tremendous heterogeneity both between tumors in different individuals and even within the tumors of a single individual. Glioblastomas also have a robust tumor initiating cell (TIC, a.k.a. cancer stem cell) compartment that is one of the best studied TIC compartments in all of oncology research. The failure to kill these TICs means that tumors will inevitably recur after treatment. As TICs are even more resistant to traditional cancer therapies than are the other cells of the tumor, it is particularly difficult to eliminate the TICs.

Moreover, our studies on glioblastoma cells demonstrated that these tumor cells inhibited c-Cbl function by formation of a complex with Cool-1/βpix.

GBMs are also of interest as many general principles of cancer biology have been identified by studying these cancers. The combination of being able to discover general principles and the extraordinary difficulty in treating GBMs suggest that success with treatment of these tumors has a high likelihood of providing therapeutic approaches of broader value in the treatment of other gliomas, other brain tumors, and tumors occurring in other parts of the body. As an example of the therapeutic efficacy of DLCTD-based therapies, immunodeficient NSG (NOD− Scid− gamma) mice were transplanted intrancranially with 500,000 luciferase-expressing human GBM cells, derived from three different GBM patients. In all experiments, tumors were allowed to grow until they were clearly visible by non-invasive imaging with an Advanced Molecular Imager (AMI; Spectral Instruments), which generally took two-three weeks. Mice were then assigned to different treatment groups, with a distribution that ensured equal representation of different tumor sizes in each group.

Mice were treated via oral gavage to administer treatment, as MPT, TMX and TMZ are all orally available. Mice treated with saline showed continuous tumor growth and died at nine weeks, at which time all treatments were stopped. TMZ treatment caused a transient suppression of tumor growth but tumor growth was evident by the seventh week of treatment. This is an outcome that is essentially identical to treatment of patients, for whom tumor recurrence is frequently seen during treatment of TMZ. TMZ-treated mice lived 4-5 weeks after treatment was stopped.

In contrast, treatment with MPT+TMX+TMZ showed complete suppression of tumor growth throughout the time of treatment (and in some cases, caused clear decreases in photon flux detected by luciferase analysis). Moreover, mice treated with MPT+TMX+TMZ showed a markedly enhanced survival post-treatment, and lived an additional 5 months before dying from tumor recurrence. In these mice, the first evidence of tumor growth was seen ≥5 weeks after cessation of treatment, if at all, and tumors grew slowly (an outcome that would be consistent with decreases in TIC number). For one of the glioblastoma cell lines examined, there was no evidence of tumor regrowth even 3 months after the cessation of treatment. Most importantly, survival was now markedly longer (p<0.001) than seen with TMZ treatment by itself, and even the treatment for a nine week period doubled survival over that seen with TMZ alone Survival was greatly enhanced by treatment with [MPT+TMX+TMZ]. TMZ-treated mice lived 2-6 weeks after treatment was stopped (FIG. 15). In contrast, mice treated with [MPT+TMX+TMZ] showed a markedly enhanced survival post-treatment, and lived an additional 5 months before dying from tumor recurrence. Average survival was twice long as seen with TMZ treatment by itself (p<0.001), despite TMZ being the first-line drug for GBM treatment and the current experiments representing the beginning stages of treatment optimization. More importantly, lifespan beyond that achieved by treating animals only with saline was extended by 5-fold more was achieved by treatment with temozolomide. These benefits were obtained even though the temozolomide dosage used by only ˜25% of the lowest standard dosages used in the clinic (as determined by FDA-provided body surface area equivalence calculations).

Treatment with MPT+TMX thus increased the sensitivity of GBM cells to temozolomide, an alkylating agent that represents the current frontline pharmacological treatment for GBM. Thus, this treatment not only enhanced susceptibility to tamoxifen but also increased the sensitivity to the frontline agent used in the treatment of glioblastoma. In addition, we do not observe the gross CNS damage expected from nine weeks of treatment with anti-cancer agents was not seen in mice treated with all three agents.

### Example 20

The ability of treatment with a CRA/DCLTD combined with a pro-oxidant to suppress tumor growth and to increase sensitivity to standard anticancer agents is not limited to treatment of glioblastomas and is not limited to increasing the vulnerability of glioblastoma cells to temozolomide.

Another example of the general utility of treatment with a CRA/DCLTD with a pro-oxidant to offer clinically relevant benefits is offered by studies of luminal breast cancer cells selected in vitro to be resistant to treatment with TMX.

In our studies on TMX-resistant luminal breast cancer cells, we first focused on cyclophosphamide (CPP), an alkylating agent used in treating TMXR breast cancer. We found selection for TMX resistance also caused increased resistance to 4-hydroxycyclophosphamide (4^{OH}-CPP, a toxic CPP metabolite generated in vivo), such that 48 hrs of exposure to 5 μM 4^{OH}-CPP killed 70% of parental MCF-7 cells but <10% of TMXR MCF-7 cells. Resistance to 4^{OH}-CPP was eliminated by co-exposure to MPT+TMX. For example, adding 3 μM MPT+5 μM TMX+5 μM 4^{OH}-CPP eliminated all TMXR MCF-7 cells within 48 hrs (a more effective cell elimination than seen with 5 days exposure to 3 μM MPT+5 μM TMX). In contrast, adding MPT or TMX individually only slightly increased vulnerability to 4^{OH}-CPP (data not shown). We started in vivo studies with a CPP dose of 3 mg/kg (which is considerably below common murine doses of 10-100 mg/kg and represents a human dose equivalent of ˜0.25 mg/kg (based on FDA body surface area conversion guidelines), about 25% of standard human low dose oral usage). This starting dose was chosen based on concerns with developing treatments less toxic than existing approaches, and on predictions of the effects of MPT+TMX treatment-induced decreases in levels and/or activity of proteins important in chemoresistance. MPT and TMX were applied at clinically relevant dosages. For MPT (35 mg/kg), the daily clinical dose in mice is 15-30 mg/kg (maximum=45 mg/kg). For TMX (4.2 mg/kg), the mouse equivalent of the daily dose for standard LBC treatment is 4-7 mg/kg, and would be increased for high dose applications. To compare outcomes with our findings on GBM cells, mice in this first group were treated with saline, TMX, [MPT+TMX], or [MPT+TMX+CPP] (oral gavage, 5 days on/2 days off). Immune-deficient NSG mice were transplanted with 500,000 luciferase-expressing human TMXR MCF7 cells in the mammary fat pads. After tumors were visible by non-invasive imaging (generally 1 to 2 weeks), mice were assigned to treatment groups, with equal representation of tumor sizes in each group.

FIG. 16 demonstrates the benefit of treatment with the novel approach of the present invention. Tumors grew rapidly in saline-treated mice (A), and also in TMX-treated mice (B), thus confirming the tamoxifen resistance of these tumors. Treatment with MPT+TMX suppressed tumor growth (C), demonstrating that the effects of treatment with the combination of a CRA/DCLTD and TMX differs from markedly from the effects of treatment with TMX alone. Treatment with the combination of MPT+TMX+CPP completely suppressed tumor growth in vivo (D), despite the fact that CPP levels were only a fraction of the lowest standard doses used in the clinic. Suppression of growth was not simply due to the effects of CPP, as treatment with CPP alone already have initiated tumor growth midway through treatment (E).

Treatment effects of MPT+TMX+CPP seem so be durable, as predicted from the observations that this treatment effectively eliminates tumor initiating cells. FIG. 16F shows outcomes at 5 weeks after the cessation of treatment, with no tumor recurrence. These observations raise the possibility that this treatment is able to control the growth of a type of breast cancer, resistant to hormone therapies, for which current therapies are inadequate.

### Example 21

As already discussed in relation to GBM cells, restoration of c-Cbl function can decrease levels and/or activity of multiple proteins important in cancer cell growth. This outcome is a general characteristic of restoring c-Cbl function in cancer cells in which c-Cbl is inhibited, as indicated also by studies on TMX-resistant LuBC cells treated with MPT+TMX (FIG. 17). MPT+TMX decreases levels of HER2, EGFR, and β-catenin as examples of direct c-Cbl targets. Examples of indirect targets of c-Cbl that also are decreased by treatment of TMX resistant LuBC cells with MPT+TMX include decreased levels of phosphorylated Erk1/2 (not shown) and Akt (FIG. 17), two downstream targets of activated RTKs that decrease in activity when c-Cbl is activated via the RFC pathway. Moreover, as seen following treatment of glioblastoma cells with MPT+TMX, we also found decreased levels of Sox2 (not shown), a transcription factor critical in TIC function. All outcomes required c-Cbl function, and were prevented by c-Cbl knockdown. Moreover, all outcomes required treatment with MPT+TMX, and were not caused by treatment with but either drug alone.

Treatment of TMX-resistant luminal breast cancer cells with MPT+TMX (but not with either agent on its own) also caused c-Cbl dependent decreases in levels of integrin β1, snail and slug, three markers of the epithelial-to-mesenchymal transition (EMT) that is associated with acquired TMX resistance.

### Example 22

The ability of CRA/DCLTD-based therapies, combined with RFC-pathway mediated activation of c-Cbl to cause decreases in levels of multiple c-Cbl targets in multiple cancers, and with different types of CRAs/DCLTDs, is supported by studies on the effects of treatment with sertraline (SRT)+TMX on c-Cbl and two c-Cbl targets in BLBC cells (FIG. 18). Levels of phosphorylated c-Cbl were increased by treatment with HER2, Notch-1 and beta-catenin levels and activation of the indirect target (p-AKT) fare decreased by treatment with 5 μM MPT+5 μM TMX for 6 hours in scrambled knockdown cells. This effect was abolished in c-Cbl knockdown cells.

### Example 23

Detection of cells or tumors in which treatment with the approaches of the present invention is indicated can be achieved by multiple means, as discussed in later sections of this document. Here evidence is provided that staining of cells with an antibody against a phosphorylated serine residue on Cool-1/βpix can be used to identify glioblastoma cells in which c-Cbl is inhibited by complex formation with Cool-1/βpix (FIG. 19A). Treatment with MPT (which decreases the levels of the inhibitory complex between c-Cbl and Cool-1/βpix) decreases the level of staining with this antibody directed against Serine340 of Cool-1/βpix (FIG. 19B).

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Throughout this application, various publications are referenced. The disclosures of these publications in their entireties are hereby incorporated by reference into this application in order to more fully describe the state of the art to which the methods and systems pertain.

It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

1. A method of treating cancer in a patient in need thereof, comprising:
(a) determining whether the cancer include cells having decreased or inhibited c-Cbl function;
(b) administering at least one agent in an amount effective to restore c-Cbl function;
(c) administering at least one agent to oxidize said cells having decreased or inhibited c-Cbl function, thereby activating c-Cbl via the redox/Fyn/c-Cbl pathway; and
(d) providing cancer therapy to the patient.

2. The method according to claim 1, wherein the agent that restores c-Cbl function comprises a CRA.

3. The method according to claim 1, wherein the agent that restores c-Cbl function also worsens lysosomal function.

4. The method of claim 1, wherein the agent that restores c-Cbl function comprises estradiol propionate, neomycin, metoprolol, methylprednisolone, dequalinium, maprotiline, meclizine, naloxone, mechlorethamine, celastrol, carboplatin, acetyltryptophan, azadirachtin, proadifen, piracetam, cyclosporine, azacitidine, simvastatin, fluphenazine, monensin, mefloquine, perphenazine, sertraline, mixtures thereof, or a pharmaceutically acceptable salt thereof.

5. The method of claim 1, further comprising administering at least one agent that worsens lysosomal function.

6. The method of claim 1, wherein the agent that oxidizes said cancer cells comprises tamoxifen or carmustine.

7. The method of claim 1, wherein the cancer therapy comprises surgery, radiation therapy, chemotherapy, immunotherapy, hormone therapy, stem cell transplantation, or a combination thereof.

8. The method of claim 1, wherein the cancer therapy comprises an alkylating agent, irradiation, or a combination thereof.

8. The method of claim 1, wherein the cancer therapy comprises tamoxifen, carmustine, temozolomide, cyclophosphamide, irradiation, or a combination thereof.

9. The method of claim 1, wherein the cancer comprises melanoma, lymphoma, basal-like breast cancer, luminal breast cancer, glioblastoma, glioma, pancreatic cancer, ovarian cancer, lung cancer, head and neck cancer, bladder cancer, colon cancer, prostate cancer, or esophageal cancer cells.

10. The method of claim 1, wherein the administration steps (b) and (c) are conducted prior to initiating cancer therapy (d).

11. The method of claim 1, wherein the administration steps (b) and (c) are conducted concurrently with cancer therapy (d).

12. A method of treatment of claim 1 wherein said decreased or inhibited c-Cbl function is determined by at least one of:
(a) isolation of tumor biopsies or tumor cells; and analysis by co-immunoprecipitation of c-Cbl to determine proteins that exist in a complex with c-Cbl;
(b) analysis of tumor specimens or tumor cells with antibodies that detect modifications of c-Cbl inhibiting proteins indicating a complex with c-Cbl;
(c) analysis of gene expression patterns in tumor specimens or tumor cells to detect changes in gene expression indicative of c-Cbl inhibition.

13. A method of killing cancer stem cells (tumor initiating cells), comprising administering to a patient in need thereof:
(a) at least one agent that restores c-Cbl activity;
(b) at least one agent that oxidizes cancer cells thereby activating c-Cbl via the redox/Fyn/c-Cbl pathway; and
(c) a chemotherapeutic agent.

14. The method of claim 13, wherein the agent that restores c-Cbl function comprises estradiol propionate, neomycin, metoprolol, methylprednisolone, dequalinium, maprotiline, meclizine, naloxone, mechlorethamine, celastrol, carboplatin, acetyltryptophan, azadirachtin, proadifen, piracetam, cyclosporine, azacitidine, simvastatin, fluphenazine, monensin, mefloquine, perphenazine, sertraline, mixtures thereof, or a pharmaceutically acceptable salt thereof.

15. The method of claim 13, further comprising administering at least one agent that worsens lysosomal function.

16. The method of claim 13, wherein the agent that oxidizes said cancer cells comprises tamoxifen or carmustine.

17. The method of claim 13, wherein the cancer therapy comprises tamoxifen, carmustine, temozolomide, cyclophosphamide, irradiation, or a combination thereof.

18. A method for ex vivo purging of cancer stem cells from a sample of bone marrow cells, comprising the steps of:
(a) harvesting bone marrow cells from a mammal;
(b) treating the harvested bone marrow cells with at least one agent that restores c-Cbl activity, and at least one agent that oxidizes cancer cells thereby activating c-Cbl via the redox/Fyn/c-Cbl pathway;
(c) implanting the treated bone marrow cells back to the mammal.

19. The method of claim 18, further comprising treating the harvested bone marrow cells with at least one chemotherapeutic agent prior to implanting the treated bone marrow cells back to the mammal.
